(19) United States
(12) Patent Application Publication
(10) Pub. No.: US 2005/0235432 A1
(43) Pub. Date: Oct. 27, 2005

(54) Composition for dyeing keratin fibers, comprising at least one alcohol oxidase, at least one oxidation dye precursor, and at least one azo, methine or azomethine cationic direct dye, and process using this composition

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/044,829

(22) Filed: Jan. 28, 2005

Related U.S. Application Data
(60) Provisional application No. 60/544,347, filed on Feb. 17, 2004.

(30) Foreign Application Priority Data
Jan. 28, 2004 (FR) 04 00776

(51) Int. Cl. A61K 31/497

(52) U.S. Cl. 8/405

(57) ABSTRACT

The present disclosure relates to a composition for dyeing keratin fibers, for instance human keratin fibers such as the hair, comprising, in a medium that is suitable for dyeing, at least one oxidation dye precursor, at least one alcohol oxidase enzyme, at least one substrate for the enzyme and at least one cationic direct dye chosen from azo, methine and azomethine cationic direct dyes. The present disclosure also relates to a process for dyeing keratin fibers, comprising applying the compositions as disclosed herein, as well as a dyeing “kit” comprising the compositions.

This application claims benefit under 35 U.S.C. §119 of U.S. Provisional Application No. 60/544,347, filed Feb. 17, 2004, and of French Application FR 04/00776, filed Jan. 28, 2004, both of which are hereby incorporated by reference.

The present disclosure relates to a composition for dyeing keratin fibers, for instance human keratin fibers such as the hair, comprising, in a medium that is suitable for dyeing, at least one oxidation dye precursor, at least one alcohol oxidase enzyme, at least one substrate for the enzyme and at least one cationic direct dye chosen from azo, methine and azomethine cationic direct dyes.

It is known practice to dye keratin fibers, such as human hair, with dye compositions comprising oxidation dye precursors, such as ortho- or para-phenylenediamines, ortho- or para-aminophenols, and heterocyclic compounds, which are generally referred to as oxidation bases. Oxidation bases, are colorless or weakly colored compounds, which, when combined with oxidizing products, may give rise to colored compounds by a process of oxidative condensation.

It is also known that the shades obtained with these oxidation bases may be varied by combining them with couplers or coloration modifiers. The coloration modifiers can be chosen for example, from aromatic meta-diamines, meta-aminophenols, meta-diphenols and certain heterocyclic compounds such as indole compounds. The variety of molecules used as oxidation bases and couplers makes it possible to obtain a wide range of colors.

The “permanent” coloration obtained as a result of oxidation dyes should be able to satisfy a certain number of requirements. Thus, it should, for example, have no toxicological drawbacks, allow shades of the desired intensity to be obtained and/or have good resistance to external agents (i.e., light, bad weather, washing, permanent waving, perspiration and rubbing). The dyes should also be able to allow white hairs to be covered and, ideally, are as unselective as possible, that is to say that they can allow the smallest possible differences in coloration to be produced over the entire length of the same keratin fiber, which is generally differently sensitized (i.e. damaged) between its end and its root.

Oxidation dyeing is generally performed in strongly alkaline medium, in the presence of hydrogen peroxide. However, the use of alkaline media in the presence Qf hydrogen peroxide can have the drawback of causing considerable degradation of the fibers, and also bleaching of keratin fibers, neither of which is always desirable.

Furthermore, this type of composition can have the drawback of having to mix the aqueous hydrogen peroxide solution and the dye support at the time of application of the composition to the keratin fibers.

The oxidation dyeing of keratin fibers may also be performed using oxidizing systems other than hydrogen peroxide, such as enzymatic systems, for instance with enzymes of the 2 electron oxidase type. For example, French Patent Application No. FR 2 769 219 describes the use of a uricase enzyme and of its uric acid substrate in oxidation dyeing to dye keratin fibers. These enzymes catalyze the oxidation of a substrate by atmospheric oxygenation to generate at least one oxidation product and also aqueous hydrogen peroxide solution. The aqueous hydrogen peroxide solution generated may be used to oxidize oxidation dye precursors and, consequently, produce the color on the hair. This system can make it possible to perform oxidation dyeing without mixing at the time of use.

More recently, French Patent Application No. FR 2 833 492 describes the use of the enzyme alcohol oxidase as the sole enzyme in an oxidation dye composition for dyeing keratin fibers. However, the dye formulations using alcohol oxidase, although they can be used under conditions that do not result in hair degradation comparable to that caused by formulations using aqueous hydrogen peroxide solution, and although they offer the possibility of being formulated all-in-one, can lead to colorations that are still insufficient as regards the homogeneity of the color, the dyeing power and the chromaticity.

European Patent Application No. EP-A-0 310 675 describes the use of oxidation dye precursors of benzenic type in combination with enzymes such as pyranose oxidase or glucose oxidase. The compositions described in this document may also comprise direct dyes. However, the colorations obtained using these compositions still can be insufficient.

Accordingly, the present disclosure relates to novel compositions for dyeing keratin fibers by oxidation dyeing, which respect the nature of the keratin fiber, offer the possibility of being formulated all-in-one and/or lead to strong, homogeneous colors and high chromaticity.

The present inventor has discovered that novel compositions comprising at least one oxidation dye precursor, at least one enzyme of alcohol oxidase type, at least one substrate for the enzyme and at least one cationic direct dye chosen from azo, methine and azomethine cationic direct dyes can produce chromatic, strong, unselective and fast colors. These compositions can give varied significant shades of intense and uniform color, without any significant degradation of the hair.

Other characteristics, aspects, subjects and benefits of the present disclosure will emerge even more clearly upon reading the description and the examples that follow.

For the purposes of the present disclosure, the following definitions apply:
azo dye is a molecule or a molecule residue that absorbs light radiation in the visible range (400 to 750 nm) and that comprises in its structure at least one sequence (A) not included in a ring:
   —N═N— (A)
methine dye is a molecule or a molecule residue that absorbs light radiation in the visible range (400 to 750 nm) and that comprises in its structure at least one sequence (B) not included in a ring
   —C═C— (B)
azomethine dye is a molecule or a molecule residue that absorbs light radiation in the visible range (400 to 750 nm) and that comprises in its structure at least one sequence (C) not included in a ring
   —N═C— (C)

Among the azo, methine and azomethine cationic direct dyes that may be used herein, non-limiting mention can be made of the following compounds:
The compounds of formula (I): 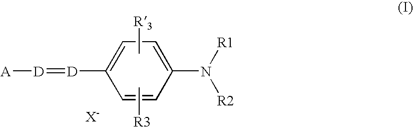
wherein D is chosen from a nitrogen atom and CH groups,
   R₁ and R₂, which may be identical or different, are chosen from hydrogen atoms, C₁-C₄ alkyl radicals optionally substituted with a CN, OH or NH₂ radical, or form, with a carbon atom of the benzene ring, a nitrogen heterocycle optionally also comprising an oxygen atom or a nitrogen atom, which may be substituted with at least one C₁-C₄ alkyl radical; R₁ and R₂ may also form a 4′-aminophenyl radical;
   R₃ and R₁₃, which may be identical or different, are chosen from hydrogen atoms, halogen atoms chosen from chlorine, bromine, iodine and fluorine, cyano radicals, C₁-C₄ alkoxy radicals and acetyloxy radicals.

X⁻ is an anion, for example, chosen from chloride, methyl sulfate and acetate.

A is a group chosen from those of structures A1 to A18:

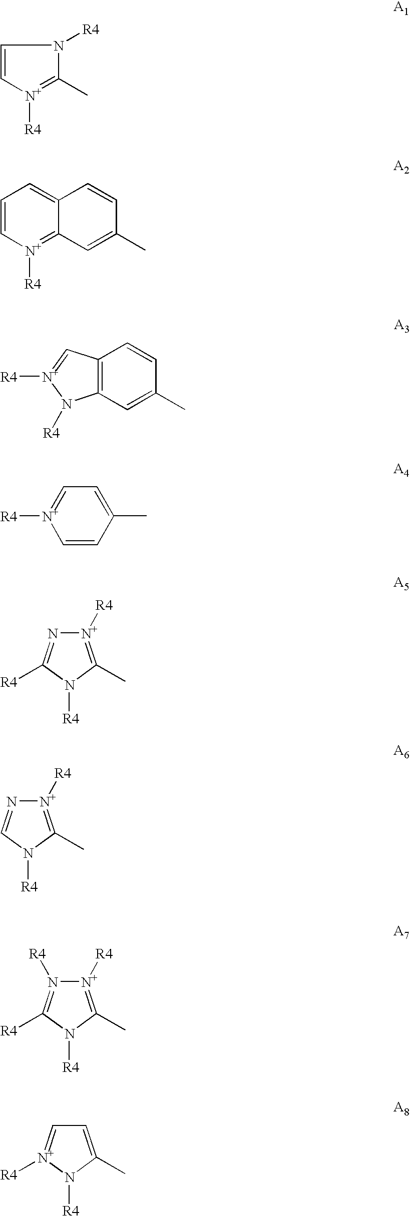

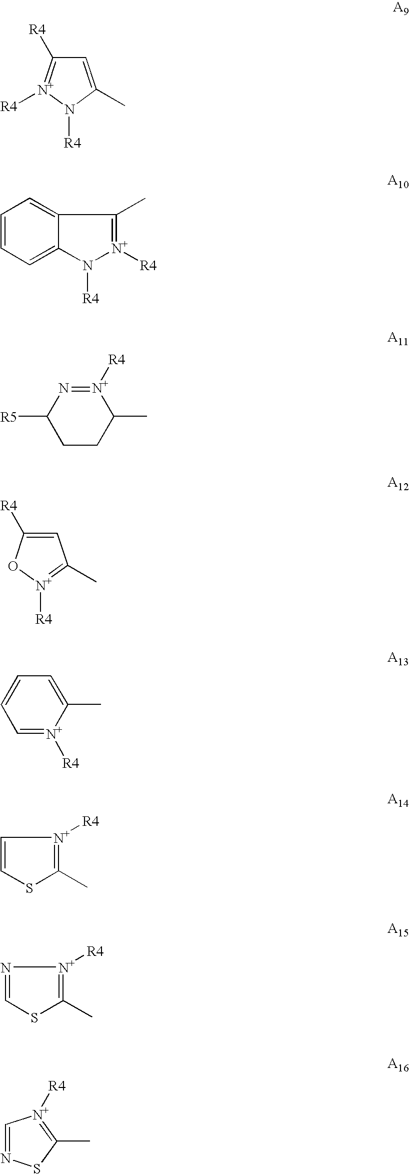

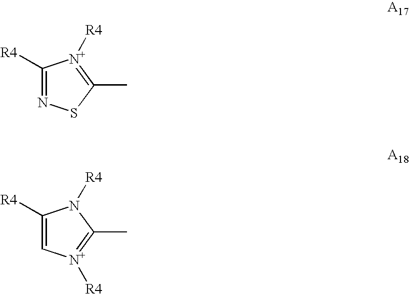

R₄ is chosen from C₁-C₄ alkyl radicals optionally substituted with a hydroxyl radical and R₅ is chosen from C₁-C₄ alkoxy radicals.
The compounds of formula (II): 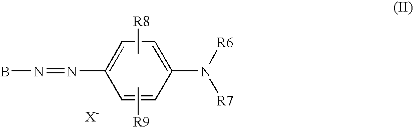
wherein R₆ is chosen from a hydrogen atom and C₁-C₄ alkyl radicals;
   R₇ is chosen from a hydrogen atom, alkyl radicals optionally substituted with a CN radical or with an amino group, and 4′-aminophenyl radicals, or forms with R₆ a nitrogen heterocycle optionally also comprising an oxygen atom and/or a nitrogen atom which may be substituted with a C₁-C₄ radical;
   R₈ and R₉, which may be identical or different, are chosen from hydrogen atoms, halogen atoms such as bromine, chlorine, iodine or fluorine, C₁-C₄ alkyl radicals, C₁-C₄ alkoxy radicals, and CN radicals;
   X⁻ is an anion, for example, chosen from chloride, methyl sulfate and acetate;
   B represents a group chosen from the structures B₁ to B₆: 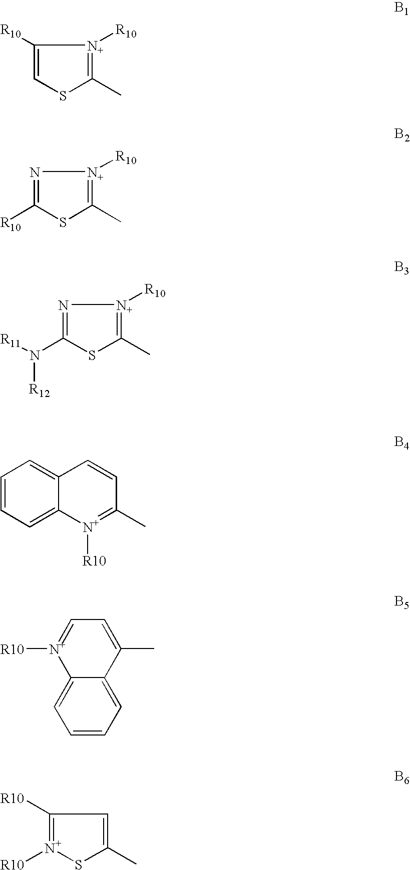
wherein R₁₀ is chosen from C₁-C₄ alkyl radicals;
   R₁₁ and R₁₂, which may be identical or different, are chosen from hydrogen atoms and C₁-C₄ alkyl radicals.
The compounds of formulae (III) and (III′): 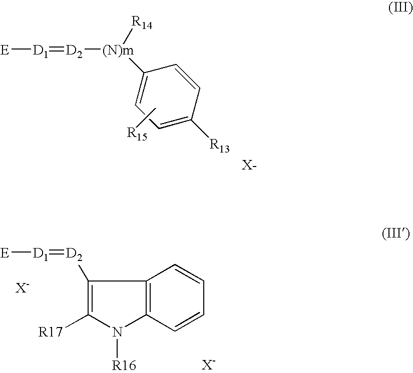
wherein, R₁₃ is chosen from a hydrogen atom, C₁-C₄ alkoxy radicals, halogen atoms such as bromine, chlorine, iodine or fluorine, and amino radicals;
   R₁₄ is chosen from a hydrogen atom, and C₁-C₄ alkyl radicals, or forms, with a carbon atom of the benzene ring, a heterocycle optionally comprising oxygen and/or nitrogen, substituted with at least one C₁-C₄ group;
   R₁₅ is chosen from a hydrogen atom and halogen atoms such as bromine, chlorine, iodine or fluorine;
   R₁₆ and R₁₇, which may be identical or different, are chosen from hydrogen atoms and C₁-C₄ alkyl radicals;
   D₁ and D₂, which may be identical or different, are chosen from nitrogen atoms and CH groups;
   m is equal to 0 or 1;
   it being understood that when R₁₃ is an unsubstituted amino group, then D₁ and D₂ simultaneously represent a —CH group and m is 0;
   X⁻ is an anion, for example chosen from chloride, methyl sulfate and acetate;
   E is a group chosen from the groups E₁ to E₈: 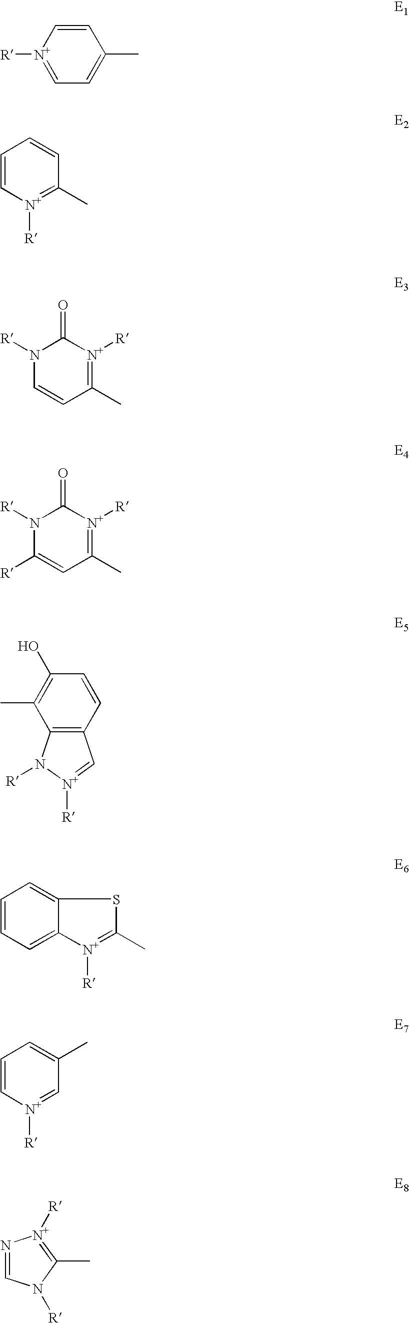
wherein R′ is chosen from C₁-C₄ alkyl radicals.

When m is 0 and D₁ is a nitrogen atom, then E may also be chosen from the group E₉: 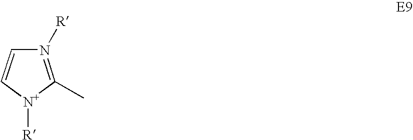
wherein R′ is chosen from C₁-C₄ alkyl radicals.

The compounds of formulae (I), (II), (III) and (III′) are described, for example, in French Patent Application No. FR 2 807 650, the content of which is incorporated herein by reference.
The compounds of formula (IV):
   W₁—W₂—N═N—W₃-L-W₄—N═N—W₅—W₆ (IV)
wherein W₁ and W₆, which may be identical or different, are chosen from NR′₁R′₂ radicals;
W₂ and W₅, which may be identical or different, are chosen from carbon-based, pyridyl and pyridazinyl aromatic groups of formula (IVa): 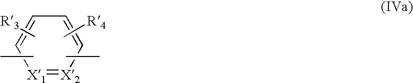
   W₃ and W₄, which may be identical or different, are chosen from heteroaromatic radicals of formulae (IVb) and (IVc): 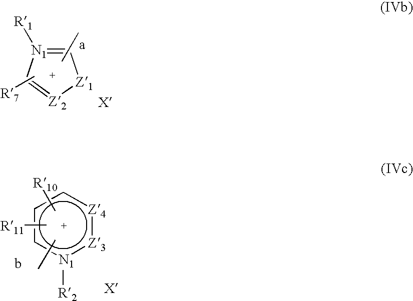
wherein X′₁ is chosen from a nitrogen atom and CR₁₅ radicals
   X′₂ is chosen from a nitrogen atom and CR′₆ radicals
   Z′₁ is chosen from oxygen and sulfur atoms and NR′₈ radicals
   Z′₂ is chosen from a nitrogen atom and CR′₉ radicals
   Z′₃ is chosen from a nitrogen atom and CR′₁₂ radicals
   Z′₄ is chosen from a nitrogen atom and CR′₁₃ radicals
   N₁ of the 5-membered ring of formula (IVb) is linked to the group L of formula (IV) via the group R′₁ and the bond a of the same 5-membered ring is linked to the azo group of formula (IV);
the bond b of the 6-membered ring of formula (IVc) is linked to the azo group of formula (IV) and N₁ of the 6-membered ring of formula (IVc) is linked to the group L of formula (IV) via the group R′₂;
   L, R′₁, R′₂, R′₃, R′₄, R′₅, R′₆, R′₇, R′₉, R′₁₀, R′₁₁, R′₁₂ and R′₁₃, which may be identical or different, are chosen from linear and branched C₁-C₁₆ hydrocarbon-based chains that can form at least one 3- to 6-membered carbon-based ring and that may be saturated or unsaturated, wherein at least one carbon of the carbon-based chain may be replaced with an oxygen, nitrogen or sulfur atom or with an SO₂ group and wherein the carbon atoms may be, independently of each other, substituted with at least one halogen atom;
   R′₁, R′₂, R′₃, R′₄, R′₅, R′₆, R′₇, R′₉, R′₁₀, R′₁, R′₁₂ and R′₁₃ can also be chosen from hydrogen atoms. L, R′₁, R′₂, R′₃, R′₄, R′₅, R′₆, R′₇, R′₉, R′₁₀, R′₁₁, R′₁₂ and R′₁₃ do not comprise any peroxide bonds or diazo or nitroso radicals and L is a divalent radical;
   R′₈ can be chosen from linear and branched C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals, and optionally substituted phenyl radicals;
   R′₇ with R′₉, R′₁₀ with R′₁₁, and R′₁₂ with R′₁₃ can also form a carbon-based aromatic ring such as a phenyl;
   X′ is chosen from organic and mineral anions.

The compounds of formula (IV) are described, for example, in French Patent Application No. FR 2 822 696, the content of which is incorporated herein by reference.
The compounds of formula (V):
   W₇—N═N—W₈—N═N—W₉ (V)
wherein:
   W₇ and W₉, which may be identical or different, are chosen from heteroaromatic radicals of formulae Va and Vb: 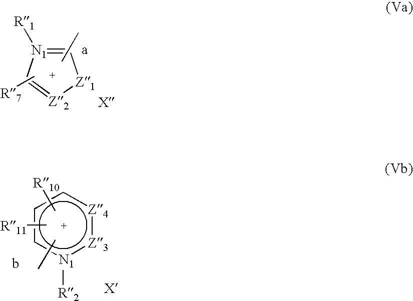
   W₈ is chosen from carbon-based, pyridyl and pyridazinyl aromatic groups of formula (Vc): 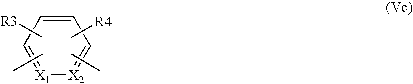
wherein:
   X₁ is chosen from a nitrogen atom and CR″₅ radicals
   X₂ is chosen from a nitrogen atom and CR″₆ radicals
   Z″₁ is chosen from oxygen and sulfur atoms and NR″₈ radicals
   Z″₂ is chosen from a nitrogen atom and CR″₉ radicals,
   Z″₃ is chosen from a nitrogen atom and CR″₂ radicals
   Z″₄ is chosen from a nitrogen atom and CR″₃ radicals
wherein the bond a of the 5-membered cationic ring of formula (Va) is linked to the azo group of formula (V);
the bond b of the 6-membered cationic ring of formula (Vb) is linked to the azo group of formula (V);
   R₃, R₄, R₁₁, R₁₂, R₁₅, R₁₆, R₁₇, R₁₈, R″₉, R″₁₀, R″₁₁, R″₁₂ and R″₁₃, which may be identical or different, are chosen from hydrogen atoms, linear and branched, saturated and unsaturated C₁-C₁₆ hydrocarbon-based chains, which can form at least one 3- to 6-membered carbon-based ring, wherein at least one carbon atom of the carbon-based chain may be replaced with an oxygen, nitrogen or sulfur atom or with an SO₂ group, and wherein the carbon atoms may be, independently of each other, substituted with at least one halogen atom;
   R₃, R₄, R″₁, R″₂, R″₅, R″₆, R″₇, R″₈, R″₉, R″₁₀, R″₁₁, R″₁₂ and R″₁₃ do not comprise any peroxide bonds or diazo or nitroso radicals;
   R″₇ and R″₉, R″₁₀ and R″₁₁, and R″₁₂ and R″₁₃ may also form a carbon-based ring, such as a phenyl;
   X′ and X″ are chosen from organic and mineral anions chosen, for example, from halides, hydroxide, sulfate, hydrogen sulfate, C₁-C₆ alkyl sulfates, acetate, tartrate, oxalate, C₁-C₆ alkyl sulfonate, and arylsulfonate, which is unsubstituted or substituted with a C₁-C₄ alkyl radical.

The compounds of formula (V) are described, for instance, in French Patent Application No. FR 2 825 702, the content of which is incorporated herein by reference.
The compounds of formulae (VIa) and (VIb): 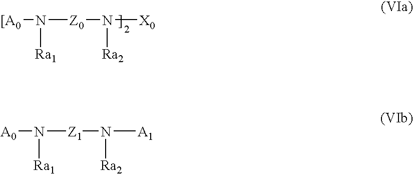
wherein A₀ and A₁, which may be identical or different, are chosen from compounds of formula (VId): 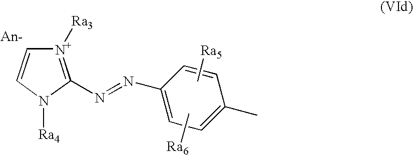
wherein
   Z₀ is chosen from aliphatic radicals
   Z₁ is chosen from alkyl radicals
   Rₐ₁ and Rₐ₂, which may be identical or different, are chosen from hydrogen atoms; and (C₁-C₄)alkyl radicals optionally substituted with at least one entity chosen from halogen atoms, hydroxyl radicals, carboxyl radicals, and cyano radicals; (C₁-C₄)alkoxy radicals optionally substituted with at least one entity chosen from hydroxyl and (C₁-C₄)alkoxy radicals; and amino, alkylamino, dialkylamino, aminocarbonyl, phenyl, phenoxy and phenylaminocarbonyl radicals, in which the phenyl radical is unsubstituted or substituted with a (C₁-C₄)alkyl, (C₁-C₄)alkoxy or phenoxy radical;
   Rₐ₁ and Rₐ₂ may also form, together with the two nitrogen atoms that bear them and the radical Z₀, a piperazine radical;
   X₀ is a bridging radical chosen from —CO—, —COCH₂—, —CH₂CO—, —COCO—, 1,4-dicarbonylphenyl and —CH₂—CH₂— radicals, or a triazine chosen from the formulae: 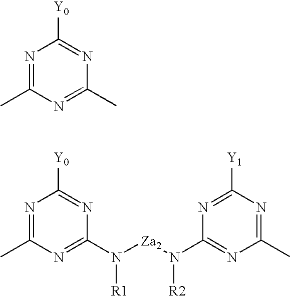
wherein Y₀ and Y₁, which may be identical or different, are chosen from halogen atoms, hydroxyl radicals, amino radicals, monoalkylamino radicals, dialkylamino radicals, 1-piperidino radicals, morpholino radicals, and 1-piperazino radicals, the piperazino radicals being unsubstituted or substituted on the nitrogen atom not attached to the triazine ring with a (C₁-C₄)alkyl radical, the (C₁-C₄)alkyl radicals being unsubstituted or substituted with hydroxyl, amino, mono(C₁-C₄)alkylamino or di(C₁-C₄)alkylamino radicals; R₁ and R₂ are chosen from hydrogen atoms and C1-C4 alkyl radicals optionally substituted with a hydroxy radical;
   Zₐ₂ is chosen from C₂-C₈ alkylene radicals or forms, with the two adjacent nitrogen atoms and the radicals R₁ and R₂, a piperazine ring;

In the radical of formula (VId), Rₐ₃ and Rₐ₄, which may be identical or different, are chosen from hydrogen atoms, (C₁-C₄)alkyl radicals, and C₁-C₄ alkyl radicals substituted with at least one entity chosen from halogen atoms, hydroxyl radicals, carboxyl radicals, cyano radicals, (C₁-C₄)alkoxy radicals, (C₁-C₄)alkoxy radicals substituted with a hydroxyl radical, (C₁-C₄)alkoxy radical, an amino, alkylamino, dialkylamino, aminocarbonyl, phenyl, phenoxy or phenylaminocarbonyl radical, in which the phenyl radical is unsubstituted or substituted with a (C₁-C₄)alkyl, (C₁-C₄)alkoxy or phenoxy radical;
Rₐ₅ and Rₐ₆, which may be identical or different, are chosen from hydrogen atoms, C₁-C₄ alkyl radicals and C₁-C₄ alkoxy radicals, optionally substituted with a hydroxyl radical, a carboxyl radical, a halogen atom, a cyano radical, a (C₁-C₄)alkoxy radical optionally substituted with a hydroxyl radical or (C₁-C₄)alkoxy radical, an amino radical, an alkylamino radical, a dialkylamino radical, an aminocarbonyl radical, a phenyl radical, a phenoxy radical or a phenylaminocarbonyl radical, in which the phenyl radical is unsubstituted or substituted with a (C₁-C₄)alkyl, (C₁-C₄)alkoxy or phenoxy radical;
An⁻ is an anion.
The compounds of formula (VIc): 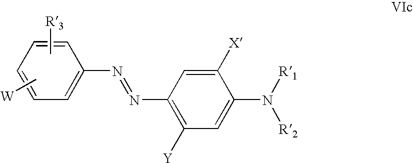
wherein the cationic charge number is 2;
   X′ and Y, which may be identical or different, are chosen from hydrogen atoms, halogens, (C₁-C₄)alkyl radicals, (C₁-C₄)alkoxy radicals, (C₁-C₄)alkylcarbonylamino radicals, arylcarbonylamino radicals, ureido radicals, and arylureido radicals;
   R′₁ is chosen from a hydrogen atom, substituted alkyl and aryl radicals, unsubstituted alkyl and aryl radicals or is the same as R′₂;
   R′₂ is chosen from radicals of formula: 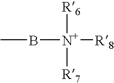
wherein B in this formula is chosen from linear and branched alkylene radicals;
   R′₆ is chosen from a hydrogen atom and substituted and unsubstituted alkyl radicals;
   R′₇ and R′₈, which may be identical or different, are chosen from substituted and unsubstituted alkyl radicals;
   R′₆ and R′₇ together with the nitrogen atom, can also form a substituted or unsubstituted 5-, 6- or 7-membered ring, which may comprise other hetero atoms, or alternatively R′₆ and R′₇ and R′₈ together can form a pyridinium ring;
   R′₃ is chosen from hydrogen and halogen atoms, and (C₁-C₄)alkyl and (C₁-C₄)alkoxy radicals;
W is a radical of formula: 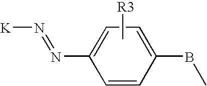
wherein K is a coupling compound chosen from those of formula (f), (g) or (h): 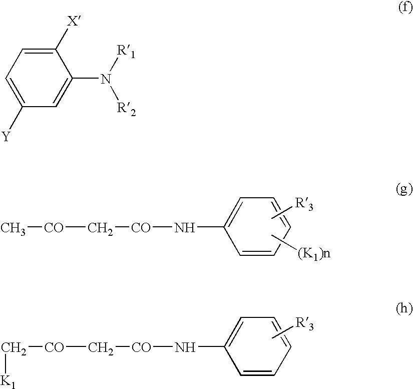
in which:
   X′ and Y, which may be identical or different, are chosen from hydrogen atoms, halogen atoms, C₁-C₄ alkyl radicals, C₁-C₄ alkoxy radicals, (C₁-C₄)alkylcarbonylamino radicals, arylcarbonylamino radicals, ureido radicals and arylureido radicals,
   R′₁ is chosen from a hydrogen atom, substituted alkyl radicals, substituted aryl radicals, unsubstituted alkyl radicals, unsubstituted aryl radicals, or has the same meaning as the substituent R′₂,
   R′₂ is chosen from radicals of formula: 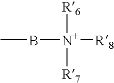
wherein B in this formula is chosen from linear and branched alkylene radicals;
   R′₆ is chosen from a hydrogen atom and substituted and unsubstituted alkyl radicals;
   R′₇ and R′₈, which may be identical or different, are chosen from substituted and unsubstituted alkyl radicals;
   R′₆ and R′₇ together with the nitrogen atom, can also form a substituted or unsubstituted 5-, 6- or 7-membered ring, which may comprise other hetero atoms, or alternatively R′₆ and R′₇ and R′₈ together can form a pyridinium ring;
   R′₃ is chosen from hydrogen and halogen atoms, and (C₁-C₄)alkyl and (C₁-C₄)alkoxy radicals;
n is equal to 1 or 2,
K₁ is chosen from —N⁺R′₆R′₇R′₈ radicals;
   R3 has the same meanings as Rₐ₃.

B in formula (W) is a bridging radical chosen from the radicals: 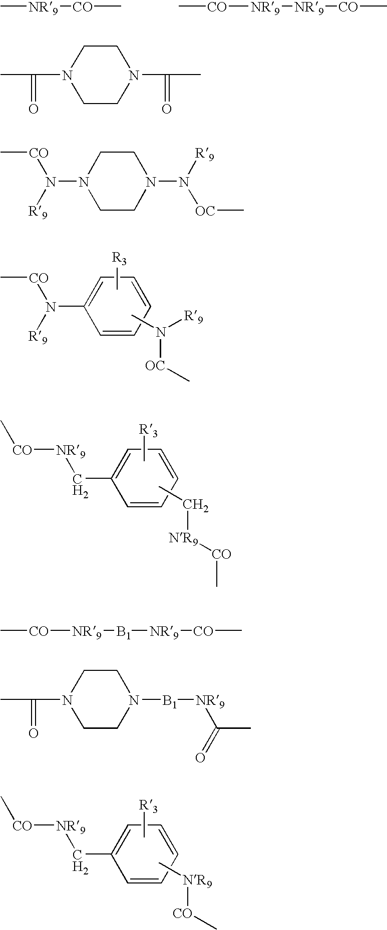
wherein R′₉ is chosen from a hydrogen atom, and C₁-C₂ alkylene radicals; B₁ is chosen from substituted and unsubstituted (C₂-C₄)alkylene radicals, the alkylene radical being linear or branched and possibly being interrupted with at least one group chosen from —NR′₉—, —O— and —S—.

The compounds of formula (VIa), (VIb) and (VIc) are described, for example, in French Patent Application No. FR 2 825 625, the content of which is incorporated herein by reference.
The compounds of formulae (VIIIa) and (VIIIb):
   Z₁-N═N-A₁-(A₃)ₙ-Z₂ (VIIIa) and Z₁-N═N-A₂ (VIIIb)
wherein n is equal to 0 or 1.
Z₁ is chosen from 5- and 6-membered cationic heteroaromatic radicals of formulae: 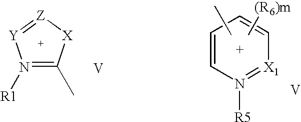
wherein X is chosen from NR₃ radicals, and S and O atoms,
Z is chosen from CR₂ radicals and N atoms, and Y is chosen from CR₄ radicals and N atoms, with the following conditions:
   when X is chosen from NR₃ radicals or O atoms and Z is a CR₂ radical, then Y is chosen from CR₄ radicals and N atoms;
   when X is a S atom, then Z or Y is a N atom; and
   when X is a S atom and Z is a N atom, then Y is a CR₄ radical.
   X₁ is chosen from CR₆ radicals and N atoms;
   m is an integer equal to 0, 1, 2 or 3.

R₁, R₃, R₅, which may be identical or different, are chosen from saturated and unsaturated, linear and branched C₁-C₁₀ hydrocarbon-based chains that can form an optionally aromatic 5- to 7-membered carbon-based ring, wherein at least one of the carbon atoms may be replaced with an oxygen, nitrogen, halogen or sulfur atom or with an SO₂ group, with the exception of the carbon linked to the nitrogen atom of the two preceding rings. The radicals R₁ and R₃ and R₅ do not comprise any peroxide bonds, or diazo, nitro or nitroso radicals.

R₂, R₄ and R₆, which may be identical or different, are chosen from hydrogen atoms, saturated and unsaturated, linear and branched C₁-C₁₀ hydrocarbon-based chains that can form an optionally aromatic 5- to 7-membered carbon-based ring; at least one of the carbon atoms optionally being replaced with at least one entity chosen from oxygen, nitrogen and sulfur atoms and SO₂ radicals, with the proviso that the radicals R₂, R₄ and R₆ do not comprise any peroxide bonds, or diazo, nitro or nitroso radicals, the radicals R₂ and R₄ together can possibly form an aromatic carbon-based ring.

V is chosen from organic and mineral anions.
A₁ and A₃, which may be identical or different, are chosen from divalent radicals of formulae (VIIIc) and (VIIId): 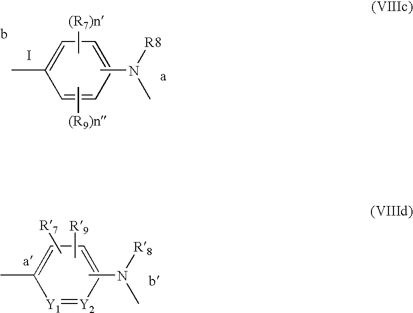
wherein
   n′ is an integer equal to 0, 1, 2 or 3.
   n″ is an integer equal to 0 or 1.
   Y₁═Y₂ is chosen from C═N and N═N;
when n is 0, then either the bond “a” of the group A₁ of formula (VIIIc) is linked to the function Z₂ of formula (VIIIa), or the bond “b′” of the group A₁ of formula (VIIId) is linked to the function Z₂ of formula (VIIIa)
when n is 1, the compounds of formula (VIIIa) are chosen from the following four structures:
   Z₁-N═N-(VIIIc)-(VIIId)-Z₂
   Z₁-N═N-(VIIIc)-(VIIIc)-Z₂
   Z₁-N═N-(VIIId)-(VIIIc)-Z₂
   Z₁-N═N-(VIIId)-(VIIId)-Z₂
wherein the divalent radicals (VIIIc) and (VIIId) are linked together or to Z₂ or to the nitrogen atom of the azo group via the linkers a, a′, b and b′.

R₈ and R′₈, which may be identical or different, are non-cationic groups chosen from hydrogen atoms, linear and branched C₁-C₁₀ hydrocarbon-based chains which can form an optionally aromatic 5- to 7-membered carbon-based ring, wherein at least one of the carbon atoms of the hydrocarbon-based chain can optionally be replaced with at least one entity chosen from oxygen, nitrogen and sulfur atoms and SO₂ groups, with the exception of the carbon linked to the nitrogen atom. The radicals R₈ or R′₈ do not comprise any peroxide bonds, or diazo, nitro or nitroso radicals;
R₇, R₉, R′₇ and R′₉, which may be identical or different, are chosen from non-cationic groups as defined for R₂ and cationic Z₃ groups, on condition that only one of the groups R₇, R₉, R′₇ and R′g is cationic;
R₇ with R₈ or, respectively, R′₇ with R′₈ can together form a saturated 5- or 6-membered heterocycle;
Z₃ is a cationic group chosen from those of formula (VIIIe):
   —(B)_{n′″}-D (VIIIe)
wherein:
B is chosen from linear and branched hydrocarbon-based chains comprising from 1 to 15 carbon atoms, which can form at least one optionally aromatic 3- to 7-membered ring, and wherein at least one of the carbon atoms of the ring(s) may be replaced with an oxygen, nitrogen or sulfur atom or with an SO₂ radical, with the exception of the carbon linked to the nitrogen atom, with the proviso that B does not comprise any peroxide bonds, or any diazo, nitro or nitroso radicals;
the radical B is linked to D via any of the atoms of the radical D;
   n″′ is equal to 0 or 1;
D is chosen from the cationic groups of formulae (VIIIf) and (VIIIg): 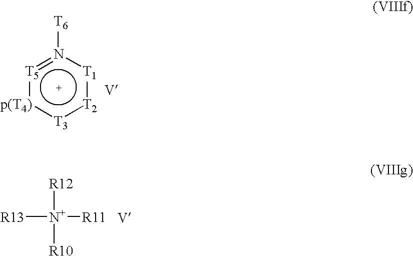
wherein:
   p is equal to 0 or 1;
   T₁, T₂, T₃ and T₄, which may be identical or different, are chosen from oxygen atoms, sulfur atoms, nitrogen atoms unsubstituted or substituted with a radical R₁₄, and carbon atoms unsubstituted or substituted with one or two radicals R₁₄, which may be identical or different;
   T₅ is chosen from nitrogen atoms and carbon atoms that are unsubstituted or substituted with a radical R₁₄;
   T₆ is chosen from linear and branched, optionally aromatic hydrocarbon-based chains comprising from 1 to 10 carbon atoms, and at least one of the carbon atoms of which may be replaced with an entity chosen from oxygen, nitrogen and sulfur atoms and SO₂ groups, and at least one of the carbon atoms of which may be, independently of each other, substituted with at least one halogen atom, with the proviso that the radical does not comprise any peroxide bonds or any diazo, nitro or nitroso radicals;
   T₁ or T₅ may also form with T₆ a 5- to 7-membered saturated or unsaturated ring, each ring member being unsubstituted or substituted with one or two radicals R₁₄, which may be identical or different;
   two of the adjacent radicals T₁, T₂, T₃, T₄ and T₅ may also form a 5- to 7-membered ring, each ring member being independently chosen from carbon atoms that are unsubstituted or substituted with one or two radicals R₁₄, which may be identical or different; nitrogen atoms that are unsubstituted or substituted with a radical R₁₄; and oxygen and sulfur atoms;
   R₁₀, R₁₁, R₁₂, R₁₃ and R₁₄, which may be identical or different, are chosen from hydrogen atoms, linear and branched, optionally aromatic hydrocarbon-based chains comprising from 1 to 10 carbon atoms, and at least one of the carbon atoms of which may be replaced with an entity chosen from oxygen, nitrogen and sulfur atoms and SO₂ groups, and at least one of the carbon atoms of which may be, independently of each other, substituted with at least one halogen atom, with the proviso that the radical does not comprise any peroxide bonds or any diazo, nitro or nitroso radicals;
   R₁₀, R₁₁, and R₁₂ may also form, in pairs with the quaternary nitrogen atom to which they are attached, at least one saturated 5- to 7-membered ring, each ring member being independently chosen from carbon atoms that are unsubstituted or substituted with one or two radicals R₁₄, which may be identical or different; nitrogen atoms that are unsubstituted or substituted with a radical R₁₄ and oxygen and sulfur atoms;
   when n″′ is equal to 0, then the group of formula (VIIIg) may be linked to the compound of formula (VIIIc) and (VIIId) directly via the nitrogen atom of the quaternary ammonium, R₁₃ being in this case a single bond;
   V′ is chosen from organic and mineral anions;
   Z₂ is chosen from linear and branched C₁-C₁₀ hydrocarbon-based chains that can form an optionally aromatic 5- to 7-membered carbon-based ring, wherein at least one of the carbon atoms may be replaced with at least one entity chosen from oxygen, nitrogen and sulfur atoms and SO₂ groups; with the proviso that the radical Z₂ does not comprise any peroxide bonds, or diazo, nitro or nitroso radicals; and cationic Z₃ groups as defined above, with the proviso that Z₂ is not cationic when R₇, R₉, R_{7′} or R_{9′} is cationic;
A₂ is chosen from radicals of formula (VIIIh) which are chosen from carbon-based, pyridyl and pyridazinyl aromatic radicals substituted with a 5-membered cationic heteroaromatic radical, optionally substituted with at least one R₁₉ radical, which has the same definition as R₂, and radicals of formula (VIIIi): 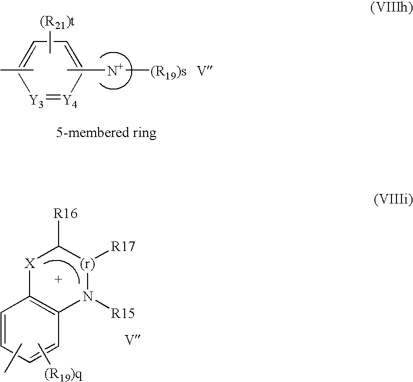
wherein:
   r is an integer equal to 0 or 1.
   q is an integer equal to 0, 1, 2 or 3.
   s is an integer equal to 0, 1, 2, 3, 4 or 5.
   t is an integer equal to 0, 1 or 2.
   Y₃═Y₄ is chosen from C═C, C═N and N═N radicals.
   if r=0, then X is chosen from O and S atoms, and NR₁₈ and CR₂₀ radicals.
   if r=1, then X is chosen from CR₂₀ radicals.
   R₁₅ and R₁₈ have the same definition as R₁ defined above.
   R₁₆, R₁₇, R₁₉, R₂₀ and R₂₁ have the same definition as R₂ defined above.
   V″ is chosen from organic and mineral anions with the condition that in formula (VIIIa), one of the groups A₁, Z₂ and A₃ is a cationic group.

The compounds of formula (VIIIa) and (VIIIb) are described, for example, in French Patent Application No. FR 2 822 698, the content of which is incorporated herein by reference.
The compounds of formula (IX):
   W₁—N═N—W₂—W₃ (IX)
wherein W₁ is chosen from 5-membered cationic aromatic heterocycles of formula (IXa): 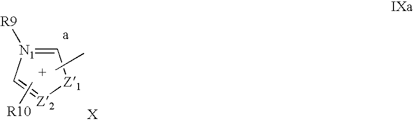
W₂ is chosen from carbon-based and pyridyl aromatic divalent groups of formula (IXb) and (IXc): 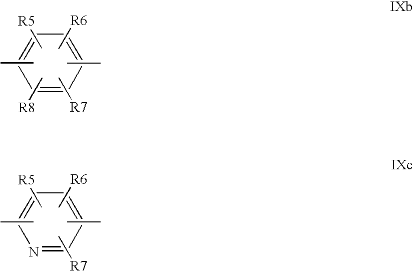
W₃ is chosen from 5- and 6-membered heterocycles of formula (IXd): 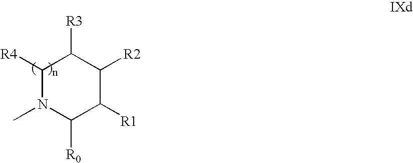
wherein:
   Z′₁ is chosen from oxygen and sulfur atoms and NR₁₂ radicals.
   Z′₂ is chosen from nitrogen atoms and CR₁₁ radicals.
   R₉ and R₁₂, which may be identical or different, are chosen from C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl, and sulfonic radicals, and optionally substituted phenyl radicals;
   R₁₀ and R₁₁, which may be identical or different, are chosen from hydrogen atoms; C₁-C₄ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl, sulfonic, and optionally substituted phenyl radicals; carboxyl radicals and sulfonylamino radicals.

R₅, R₆, R₇ and R₈, which may be identical or different, are chosen from hydrogen atoms, chlorine atoms, bromine atoms, linear and branched C₁-C₆ hydrocarbon-based chains, which can form at least one 3- to 6-membered carbon-based ring, and which may be saturated or unsaturated, wherein at least one of the carbon atoms of the carbon-based chain may be replaced with an entity chosen from oxygen, nitrogen and sulfur atoms and SO₂ groups, and wherein the carbon atoms may be, independently of each other, substituted with at least one halogen atom; with the proviso that R₅, R₆, R₇ and R₈ do not comprise any peroxide bonds or diazo or nitroso radicals;
n is an integer equal to 0 or 1;
R₀, R₁, R₂, R₃ and R₄, which may be identical or different, are chosen from hydrogen atoms, hydroxyl, amino and acetoxy radicals and —NR₁₃R₁₄ radicals, wherein R₁₃ and R₁₄, which may be identical or different, are chosen from hydrogen atoms; C₁-C₄ alkyl radicals substituted with at least one entity chosen from halogen atoms, hydroxyl, C₁-C₂ alkoxy, amino, and C₁-C₂ amino(di)alkyl radicals; sulfonylamino radicals, carboxyl radicals, carboxamido radicals, amido radicals, mono- and dialkylamido radicals; halogen atoms; and C₁-C₆ alkyl radicals substituted with at least one radical chosen from hydroxyl, C₁-C₂ alkoxy, C₂-C₄ (poly)hydroxyalkoxy, amino and C₁-C₂ (di)alkylamino radicals, it being understood that at least one of the groups R₀, R₁, R₂, R₃ and R₄ is other than hydrogen;
X is chosen from organic and mineral anions.

The compounds of formula (IX) described, for example, in French Patent Application No. FR 2 822 693, the content of which is incorporated herein by reference.
The compounds of formula (X)
   W′₁—N═N—W′₂—W′₃ (X)
wherein W′₁ is chosen from 5-membered cationic aromatic heterocycles of formula (Xa): 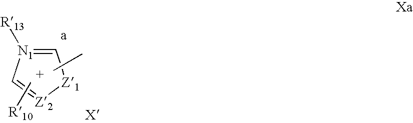
W′₂ is chosen from carbon-based and pyridyl aromatic divalent groups chosen from those of formulae (Xb) and (Xc): 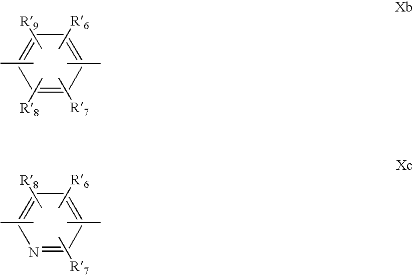
   W′₃ is chosen from 7- and 8-membered heterocycles of formula (Xd): 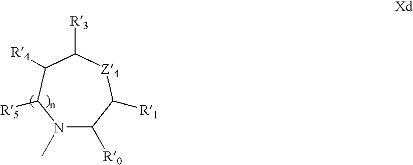
wherein:
   Z′₁ is chosen from oxygen and sulfur atoms and NR′₁₂ radicals;
   Z′₂ is chosen from a nitrogen atom and CR′₁₁ radicals;
   R′₁₂ and R′₁₃, which may be identical or different, are chosen from C₁-C₈ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; and optionally substituted phenyl radicals;
   R′₁₀ and R′₁₁, which may be identical or different, are chosen from hydrogen atoms; C₁-C₄ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; optionally substituted phenyl radicals; carboxyl radicals; and sulfonylamino radicals;
   R′₁₆, R′₁₇, R′₁₈ and R′₉, which may be identical or different, are chosen from hydrogen atoms, chlorine atoms, bromine atoms, linear and branched C₁-C₆ hydrocarbon-based chains, which can form at least one 3- to 6-membered carbon-based ring, and which may be saturated or unsaturated, wherein at least one of the carbon atoms of the carbon-based chain of which may be replaced with an entity chosen from oxygen, nitrogen and sulfur atoms and SO₂ groups, and wherein the carbon atoms of which may be, independently of each other, substituted with at least one halogen atom; with the proviso that R′₁₆, R′₁₇, R′₈ and R′₉ do not comprise any peroxide bonds or diazo or nitroso radicals;
   n is an integer equal to 1 or 2;
   Z′₄ is chosen from oxygen and sulfur atoms, NR′₁₂ radicals and CR′₂R″₂ radicals;
   R′₀, R′₁, R′₂, R″₂, R′₃, R′₄ and R′₅, which may be identical or different, are chosen from hydrogen atoms, alkyl radicals, alkoxy radicals, hydroxyl radicals, amino radicals, acetoxy radicals, and —NR₁₄R₁₅ radicals, wherein R₁₄ and R₁₅, which may be identical or different, are chosen from hydrogen atoms; C₁-C₄ alkyl radicals substituted with at least one entity chosen from halogen atoms, hydroxyl radicals, C₁-C₂ alkoxy radicals, amino radicals and C₁-C₂ amino(di)alkyl radicals; sulfonylamino radicals, carboxyl radicals, carboxamido radicals, amido radicals, mono- and dialkylamido radicals, halogen atoms, and C₁-C₆ alkyl radicals substituted with at least one radical chosen from hydroxyl, C₁-C₂ alkoxy, C₂-C₄ (poly)hydroxyalkoxy, amino and C₁-C₂ (di)alkylamino radicals;
   X′ is chosen from organic and mineral anions.

The compounds of formula (X) are described, for example, in French Patent Application No. FR 2 822 694, the content of which is incorporated herein by reference.
The compounds of formula (XI) 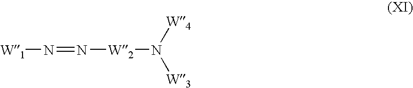
wherein W″₁ is chosen from 5-membered cationic aromatic heterocycles of formula (XIa): 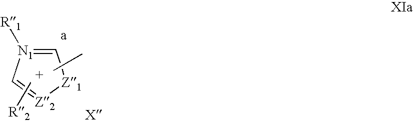
wherein
   W″₂ is chosen from carbon-based and pyridyl aromatic divalent groups of formulae (XIb) and (XIc): 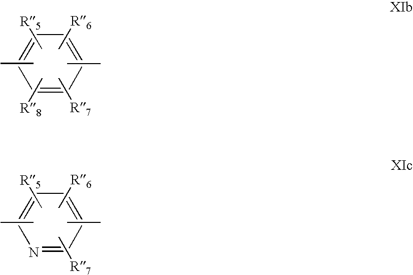
wherein:
   Z″₁ is chosen from oxygen and sulfur atoms and NR₁₄ radicals.
   Z″₂ is chosen from a nitrogen atom and CR₁₃ radicals.
   R″₁ and R″₄, which may be identical or different, are chosen from C₁-C₈ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; and optionally substituted phenyl radicals;
   R″₂ and R″₃ represent, which may be identical or different, are chosen from hydrogen atoms, C₁-C₄ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; optionally substituted phenyl radicals, carboxyl radicals and sulfonylamino radicals;
   R″₅, R″₆, R″₇, R″₈ and W″₄, which may be identical or different, are chosen from chlorine atoms, bromine atoms, linear and branched C₁-C₆ hydrocarbon-based chains, which can form at least one 3- to 6-membered carbon-based ring, and which may be saturated or unsaturated, wherein at least one of the carbon atoms of the hydrocarbon-based chain may be replaced with an entity chosen from oxygen, nitrogen and sulfur atoms and SO₂ groups, and wherein the carbon atoms of which may be, independently of each other, substituted with at least one halogen atom; with the proviso that R″₅, R″₆, R″₇, R″₈ and W″₄ do not comprise any peroxide bonds, or diazo or nitroso radicals, and W″₄ being a non-aromatic substituent;
   W″₃ is chosen from thienyl, pyrazolyl, pyrrolyl, imidazolyl, furyl, triazolyl, thiadiazolyl, isoxazolyl, 5-isothiazolyl, thiazolyl, oxazolyl, pyridyl, pyrimidinyl, triazinyl, pyridazinyl and pyrazinyl radicals, each of these heteroaromatic rings optionally being substituted with at least one C₁-C₄ alkyl radical, optionally substituted with at least one radical chosen from hydroxyl, C₁-C₄ alkoxy, (poly)hydroxyalkoxy, amino, C₁-C₄ (di)alkylamino, C₂-C₄ (poly)hydroxyalkylamino, carboxyl, sulfonyl, alkoxycarbonyl and C₁-C₄ thioether radicals; a phenyl radical optionally substituted with at least one radical chosen from C₁-C₂ alkoxy, amino, C₁-C₂ (di)alkylamino, carboxyl, sulfonyl and C₁-C₄ alkyl radicals, halogen atoms and C₁-C₂ thioether radicals; halogens such as a chlorine, fluorine or bromine atom; amino radicals; C₁-C₄ alkylamino radicals; C₂-C₄ (poly)hydroxyalkylamino radicals; C₁-C₄ (di)alkylamino radicals; C₁-C₂ alkoxy radicals; carboxyl radicals and sulfonylamino radicals;
   X″ is chosen from organic and mineral anions.

The compounds of formula (XI) are described, for example, in French Patent Application No. FR 2 822 695, the content of which is incorporated herein by reference.
The compounds of formula (XII)
   W⁰₁—W⁰₂—N═N—W⁰₃ (XII)
wherein W⁰₁ is chosen from 5-, 6-, 7- and 8-membered heterocycles of formula (XIIa): 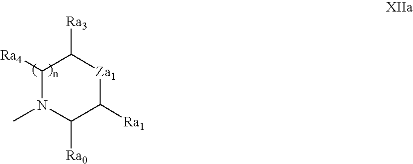
   W⁰₂ is chosen from carbon-based, pyridyl and pyridazinyl aromatic divalent groups of formula (XIIb): 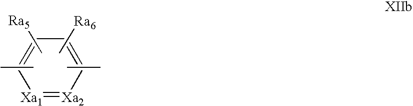
   W⁰3 is chosen from cationic heteroaromatic radicals of formula (XIIc): 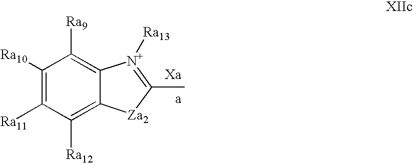
wherein:
   n is equal to 0, 1, 2 or 3, it being understood that when n is greater than or equal to 2, then the radicals Ra₄ may be identical or different;
   Xa₁ is chosen from a nitrogen atom and CRa₇ radicals;
   Xa₂ is chosen from a nitrogen atom and CRa₈ radicals;
   Za₁ is chosen from CHRa₂ radicals, oxygen and sulfur atoms, and NRa₁₄ radicals;
   Za₂ is chosen from oxygen and sulfur atoms and NRa₁₅ radicals;
   Ra₀, Ra₁, Ra₂, Ra₃, Ra₄, Ra₅, Ra₆, Ra₇, Ra₈, Ra₉, Ra₁₀, Ra₁₁, and Ra₁₂, which may be identical or different, are chosen from hydrogen atoms, linear and branched C₁-C₁₀ hydrocarbon-based chains, which can form at least one 3- to 6-membered carbon-based ring, and which may be saturated or unsaturated, wherein at least one of the carbon atoms of the carbon-based chain may be replaced with an entity chosen from oxygen, nitrogen and sulfur atoms and SO₂ groups, and wherein the carbon atoms of which may be, independently of each other, substituted with at least one halogen atom, with the proviso that Ra₀, Ra₁, Ra₂, Ra₃, Ra₄, Ra₅, Ra₆, Ra₇, Ra₈, Ra₉, Ra₁₀, Ra₁₁, and Ra₁₂ do not comprise any peroxide bonds, or diazo or nitroso radicals;
   Ra₁₄ is chosen from a hydrogen atom, linear and branched C₁-C₁₀ hydrocarbon-based chains, which can form at least one 3- to 6-membered carbon-based ring, and which may be saturated or unsaturated, at least one of the carbon atoms of the carbon-based chain of which may be replaced with an entity chosen from oxygen, nitrogen and sulfur atoms and SO₂ groups, and the carbon atoms of which may be, independently of each other, substituted with at least one halogen atom, with the proviso that Ra₁₄ does not comprise any peroxide bonds, or diazo or nitroso radicals, it being understood that the oxygen, nitrogen and sulfur atoms are not directly linked to the nitrogen atom bearing the radical Ra₁₄;
   Ra₅ and Ra₆ may also form a carbon-based aromatic ring, such as a phenyl;
   Ra₁₃ and Ra₁₅, which may be identical or different, are chosen from C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl, sulfonic and optionally substituted phenyl radicals;
   the bond a of the cationic ring of formula (XIIc) is linked to the azo group of formula (XIIa);
   Xa is chosen from organic and mineral anions.

The compounds of formula (XII) are described, for example, in French Patent Application No. FR 2 829 926, the content of which is incorporated herein by reference.

The compounds of formula (XIII)
A-N═N—B (XIII)
wherein A is a group chosen from those of formulae A₁ to A₃: 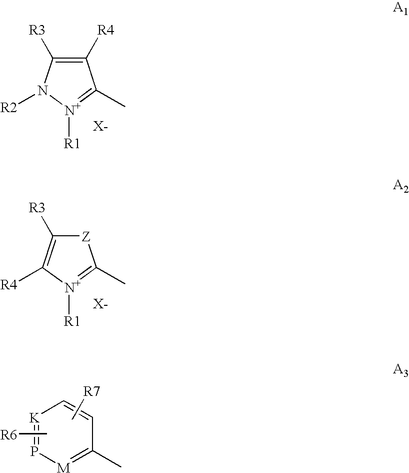
wherein R₁ is chosen from C₁-C₄ alkyl radicals, phenyl radicals which may be substituted with an entity chosen from C₁-C₄ alkyl radicals and halogen atoms chosen from chlorine, bromine, iodine and fluorine;
   R₂ is chosen from C₁-C₄ alkyl radicals and phenyl radicals;
   R₃ and R₄, which may be identical or different, are chosen from C₁-C₄ alkyl radicals, and phenyl radicals, or alternatively, in the case of the structure A₁, can together form a substituted benzene ring, and in the case of the structure A₂, can together form a benzene ring optionally substituted with at least one radical chosen from C₁-C₄ alkyl, C₁-C₄ alkoxy and NO₂ radicals;
   R₃ can also denote a hydrogen atom;
   Z is chosen from oxygen and sulfur atoms and —NR₂ radicals;
   M is chosen from —CH, —CR, wherein R is chosen from C₁-C₄ alkyl radicals, and —NR₅ (X⁻)ᵣ radicals;
   K is chosen from —CH, —CR, wherein R is chosen from C₁-C₄ alkyl radicals, and —NR₅ (X⁻)ᵣ radicals;
   P is chosen from —CH, —CR, wherein R is chosen from C₁-C₄ alkyl radicals, and —NR₅ (X⁻)ᵣ radicals,
   r is equal to 0 or 1;
   R₅ is chosen from O⁻ atoms, C₁-C₄ alkoxy radicals, and C₁-C₄ alkyl radicals;
   R₆ and R₇, which may be identical or different, are chosen from hydrogen atoms, halogen atoms chosen from chlorine, bromine, iodine and fluorine, C₁-C₄ alkyl radicals, C₁-C₄ alkoxy radicals and NO₂ radicals;
   X⁻ is an anion, for example chosen from chloride, iodide, methyl sulfate, ethyl sulfate, acetate and perchlorate, with the proviso that,
      if R₄ is chosen from C₁-C₄ alkyl radicals, and Z is a sulfur atom, then R₃ does not denote a hydrogen atom.
if R₅ is an O⁻ atom, then r is equal to zero.
   if K or P or M are chosen from —N-alkyl C₁-C₄ X⁻, then R₆ or R₇ is not a hydrogen atom.
   if K is chosen from —NR₅(X⁻)ᵣ, then M and P are the same and are chosen from —CH and —CR radicals
   if M is —NR₅(X⁻)ᵣ, then K and P are the same and are chosen from —CH and —CR radicals
   if P is —NR₅(X⁻)ᵣ, then K and M are the same and are chosen from —CH and —CR radicals
   if Z is —NR₂ and R₂ is a C₁-C₄ alkyl radical, then at least one of the radicals R₁, R₃ or R₄ of A₂ is not a C₁-C₄ alkyl radical.

B is chosen from
A groups of formula B₁: 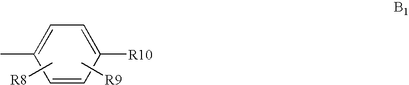
   wherein
R₈ is chosen from a hydrogen atom, halogen atoms chosen from chlorine, bromine, iodine and fluorine, C₁-C₄ alkyl radicals, C₁-C₄ alkoxy radicals, —OH radicals, —NO₂ radicals, —NHR₁₁ radicals, —NR₁₂R₁₃ radicals, and —NHCOC₁-C₄ alkyl radicals, or forms with R₉ a 5- or 6-membered ring, optionally comprising at least one hetero atom chosen from nitrogen, oxygen and sulfur atoms;
R₉ is chosen from a hydrogen atom, halogen atoms chosen from chlorine, bromine, iodine and fluorine, and C₁-C₄ alkyl and C₁-C₄ alkoxy radicals, or forms with R₁₀ or R₁₁ a 5- or 6-membered ring optionally comprising at least one hetero atom chosen from nitrogen, oxygen and sulfur atoms;
R₁₀ is chosen from a hydrogen atom, —OH radicals, —NHR1, radicals, and —NR₁₂R₁₃ radicals;
R₁₁ is chosen from a hydrogen atom, C₁-C₄ alkyl radicals, C₁-C₄ monohydroxyalkyl radicals, C₂-C₄ polyhydroxyalkyl radicals, and phenyl radicals.

R₁₂ and R₁₃, which may be identical or different, are chosen from C₁-C₄ alkyl radicals, C₁-C₄ monohydroxyalkyl radicals, and C₂-C₄ polyhydroxyalkyl radicals; or -5- and 6-membered nitrogenous heterocyclic groups that may comprise other hetero atoms and/or carbonyl groups and that may be substituted with at least one radical chosen from C₁-C₄ alkyl, amino and phenyl radicals, for example, groups of formula B₂: 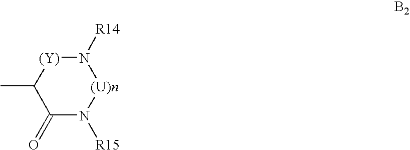
wherein, R₁₄ and R₁₅, which may be identical or different, are chosen from hydrogen atoms, C₁-C₄ alkyl radicals and phenyl radicals;
Y is chosen from —CO— radicals and 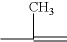
   radicals,
n is equal to 0 or 1, where, when n is equal to 1, U is a —CO radical.

The compounds of formula (XIII) are described, for example, in French Patent Application No. FR 2 807 650, the content of which is incorporated herein by reference.
The compounds of formula (XIV):
   W₁—N═N—W₂—W₃ (XIV)
wherein
   W₁ is chosen from 5-membered cationic aromatic heterocycles of formula (II): 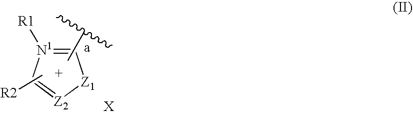
   W₂ is chosen from carbon-based and pyridyl aromatic divalent groups of formulae (IV) and (V): 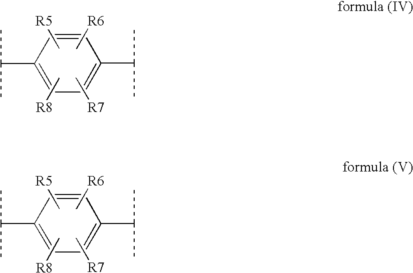
   W₃ is chosen from 5- and 6-membered nitrogenous heteroaromatic radicals linked to W₂ via the nitrogen atom of the ring of the heteroaromatic radical, the heteroaromatic radical being chosen from pyrazolyl, pyrrolyl, imidazolyl, triazolyl, thiadiazolyl, pyridazinyl and pyrazinyl radicals, each of these heteroaromatic radicals being optionally substituted with at least one entity chosen from hydrogen atoms; C₁-C₆ alkyl radicals optionally substituted with at least one radical chosen from hydroxyl, C₁-C₄ alkoxy, (poly)hydroxyalkoxy, amino, C₁-C₄ (di)alkylamino, C₂-C₄ (poly)hydroxyalkylamino, carboxyl, sulfonyl, alkoxycarbonyl and C₁-C₄ thioether radicals; and phenyl radicals optionally substituted with at least one entity chosen from halogen atoms and C₁-C₂ alkoxy, amino, C₁-C₂ (di)alkylamino, carboxyl, sulfonyl, C₁-C₄ alkyl, and C₁-C₂ thioether radicals,
   Z₁ is chosen from oxygen and sulfur atoms and NR4 radicals,
   Z₂ is chosen from a nitrogen atom CR3 radicals,
   R1 and R4, which may be identical or different, are chosen from C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl, and sulfonic radicals; and optionally substituted phenyl radicals,
   R2 and R3, which may be identical or different, chosen from hydrogen atoms; C₁-C₆ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl, and sulfonic radicals; optionally substituted phenyl radicals; carboxyl radicals; and sulfonylamino radicals;
   R5, R6, R7 and R8, which may be identical or different, are chosen from hydrogen atoms; chlorine atoms; bromine atoms; linear and branched C₁-C₆ hydrocarbon-based chains, which can form at least one 3- to 6-membered carbon-based ring, and which may be saturated or unsaturated, wherein at least one of the carbon atoms of the carbon-based chain may be replaced with an entity chosen from oxygen, nitrogen and sulfur atoms and SO₂ groups, and wherein the carbon atoms of which may be, independently of each other, substituted with at least one halogen atom; with the proviso that R5, R6, R7 and R8 do not comprise peroxide bonds, or diazo or nitroso radicals,
   X is chosen from organic and mineral anions.

The compounds of formula (XIV) are described, for example, in International Patent Application No. WO 02/078660, the content of which is incorporated herein by reference.
The compounds of formula (XV):
   W₁—N═N—W₂—(W₃)ₙ—W₄—N═N—W₅ (XV)
wherein:
n is chosen from 0 or 1,
   W₁ and W₅, which may be identical or different, are chosen from heteroaromatic radicals of formulae (II) and (III): 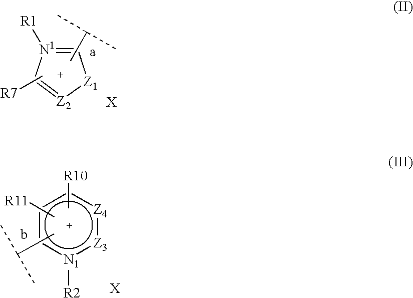
   W₃ is chosen from an oxygen atoms, NR14 radicals, —NR15-W6-NR16— radicals, —NR16-W6-O— radicals, —O—W6-O— radicals, W6 radicals and carbonyl radicals,
   W₂ and W₄, which may be identical or different, are chosen from carbon-based, pyridyl and pyridazinyl aromatic groups of formula (IV): 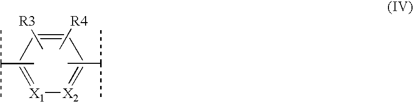
   W6 is chosen from 5- and 6-membered aromatic and heteroaromatic groups of formula (V): 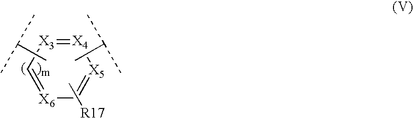
in which
   m is equal to 0 or 1,
   X₁ is chosen from a nitrogen atom and CR5 radicals,
   X₂ is chosen from a nitrogen atom and CR6 radicals,
   X₃ is chosen from nitrogen and carbon atoms, and CR18 radicals,
   X₄ is chosen from nitrogen and carbon atoms, and CR19 radicals,
   X₅ is chosen from nitrogen and carbon atoms, and CR20 radicals,
   X₆ is chosen from nitrogen and carbon atoms, and CR21 radicals,
   Z1 is chosen from oxygen and sulfur atoms, and NR8 radicals,
   Z2 is chosen from a nitrogen atom and CR9 radicals,
   Z3 is chosen from a nitrogen atom and CR12 radicals,
   Z4 is chosen from a nitrogen atom and CR13 radicals,
   the bond a of the 5-membered cationic ring of formula (II) is linked to the azo group of formula (XV),
   the bond b of the 6-membered cationic ring of formula (III) is linked to the azo group of formula (XV),
   it being understood that when X3, X4, X5, and X6 are carbon atoms, then they are linked to W₂ or W₄,
   it being understood that formula (V) comprises not more than three nitrogen atoms,
   it being understood that when formula (V) comprises three nitrogen atoms, they are not contiguous,
   R1, R2 and R8, which may be identical or different, are chosen from linear and branched C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; and phenyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals, and halogen atom such as chlorine, fluorine or bromine,
   R3, R4, R5, R6, R7, R9, R10, R11, R12, R13, R14, R15, R16, R17, R18, R19, R20 and R21, which may be identical or different, are chosen from hydrogen atoms, linear and branched C₁-C₁₆ hydrocarbon-based chains, which may be saturated or unsaturated, wherein at least one of the carbon atoms of the carbon-based chain may be replaced with an entity chosen from oxygen, nitrogen and sulfur atoms and SO₂ groups, and wherein the carbon atoms of which may be, independently of each other, substituted with at least one halogen atom; with the proviso that R3, R4, R5, R6, R7, R9, R10, R1, R12, R13, R14, R15, R16, R17, R18, R19, R20 and R21 do not comprise any peroxide bonds, or diazo or nitroso radicals,
   R7 with R9, R10 with R11 and R12 with R13 may form a carbon-based aromatic ring, such as a phenyl,
   X⁻ is chosen from organic and mineral anions.

The compounds of formula (XI) are described, for example, in International Patent Application No. WO 02/100834, the content of which is incorporated herein by reference.

The compounds of formula (XVI):
[W₁—N═N—W₂—W₃—(W₄—W₅)ₚ]₂-L (XVI)
wherein
p is equal to 0 or 1.

W₁ is chosen from heteroaromatic radicals of formulae (II) and (III):

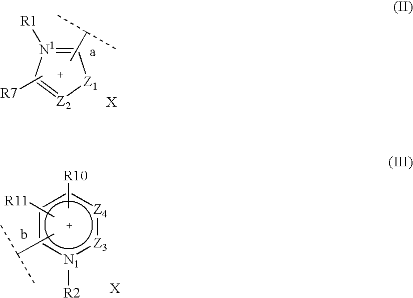

W₂ is chosen from carbon-based, pyridyl and pyridazinyl aromatic groups of formula (IV): 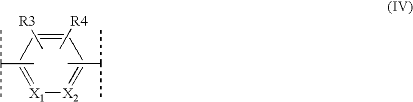
—W₃ and W₅, which may be identical or different, are chosen from —NR14— radicals, and oxygen and nitrogen atoms,
W₄ is chosen from linear and branched C₁-C₁₆ hydrocarbon-based chains, which can form at least one 3- to 6-membered carbon-based ring, and which may be saturated or unsaturated, at least one of the carbon atoms of the carbon-based chains of which may be replaced with an entity chosen from oxygen, nitrogen and sulfur atoms and SO₂ groups, and the carbon atoms of which may be, independently of each other, substituted with at least one halogen atom, with the proviso that W₄ does not comprise any peroxide bonds, or diazo or nitroso radicals; piperazine rings; and radicals of formula (V): 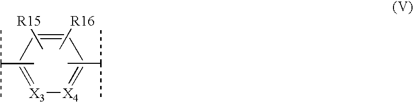
wherein:
   X₁ is chosen from a nitrogen atom and CR5 radicals
   X₂ is chosen from a nitrogen atom and CR6 radicals
   X₃ is chosen from a nitrogen atom and CR17 radicals
   X₄ is chosen from a nitrogen atom and CR18 radicals
   Z1 is chosen from oxygen and sulfur atoms and NR8 radicals,
   Z2 is chosen from a nitrogen atom and CR9 radicals,
   it being understood that (Z1, Z2) is other than (NR8, CR9),
   Z3 is chosen from a nitrogen atom and CR12 radicals,
   Z4 is chosen from a nitrogen atom and CR13 radicals,
   the bond a of the 5-membered cationic ring of formula (II) is linked to the azo group of formula (XVI),
   the bond b of the 6-membered cationic ring of formula (III) is linked to the azo group of formula (XVI),
   when p is equal to 0, then L is chosen from linear and branched C₁-C₁₆ hydrocarbon-based chains, which can form at least one 3- to 6-membered carbon-based ring, at least one of the carbon atoms of the carbon-based chain of which may be replaced with an entity chosen from oxygen, nitrogen and sulfur atoms and SO₂ groups, and the carbon atoms of which may be, independently of each other, substituted with at least one halogen atom; with the proviso that L does not comprise any peroxide bonds, or diazo or nitroso radicals,
   when p is equal to 1, then L is chosen from linear and branched C₁-C₁₆ hydrocarbon-based chains, which can form at least one 3- to 6-membered carbon-based ring, and which may be saturated or unsaturated, wherein at least one of the carbon atoms of the carbon-based chain may be replaced with an entity chosen from oxygen, nitrogen and sulfur atoms and SO₂ groups and not comprising any peroxide bonds, or diazo or nitroso radicals,
   R3, R4, R5, R6, R7, R9, R10, R11, R12, R13, R14, R15, R16, R17 and R18, which may be identical or different, are chosen from hydrogen atoms, linear and branched C₁-C₁₆ hydrocarbon-based chains, which can form at least one 3- to 6-membered carbon-based ring, and which may be saturated or unsaturated, wherein at least one of the carbon atoms of the carbon-based chain may be replaced with an entity chosen from oxygen, nitrogen and sulfur atoms and SO₂ groups, and wherein the carbon atoms of which may be, independently of each other, substituted with at least one halogen atom; with the proviso that R3, R4, R5, R6, R7, R9, R10, R11, R12, R13, R14, R15, R16, R17 and R18 do not comprise any peroxide bonds, or diazo or nitroso radicals,
   R3 with R4, R7 with R9, R10 with R11, R12 with R13 and R15 with R16 may form a carbon-based aromatic ring, such as a phenyl,
   R1, R2, and R8 are chosen from linear and branched C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; and phenyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals;
   X is chosen from organic and mineral anions.

The compounds of formula (XI) are described, for example, in International Patent Application No. WO 02/100369, the content of which is incorporated herein by reference.
The compounds of formula (XVII):
   W₁—N═N—W₂—NW₃—W₄—W₅ (XVII)
wherein
   W₁ is chosen from 5- and 6-membered cationic aromatic heterocycles of formulae (II) and (III): 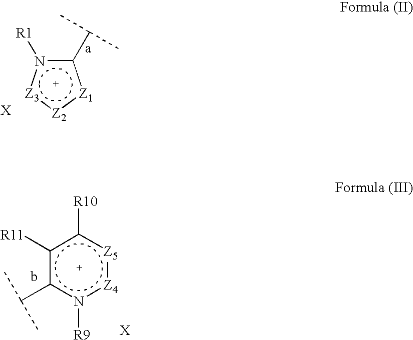
wherein:
   Z₁ is chosen from an oxygen atom, a sulfur atom, NR2 radicals and CR3 radicals,
   Z2 is chosen from a nitrogen atom and CR4 radicals,
   Z3 is chosen from NR12 radicals and CR13 radicals,
   Z4 is chosen from a nitrogen atom and CR14 radicals,
   Z5 is chosen from a nitrogen atom and CR15 radicals,
   the bond a links the 5-membered cationic ring of formula (II) to the azo function of formula (XVII).
   the bond b of the 6-membered cationic ring of formula (III) is linked to the azo function of formula (XVII).
   X is chosen from organic and mineral anions;
   W₂ and W₄, which may be identical or different, are chosen from carbon-based and pyridyl aromatic divalent groups of formulae (IV) and (V): 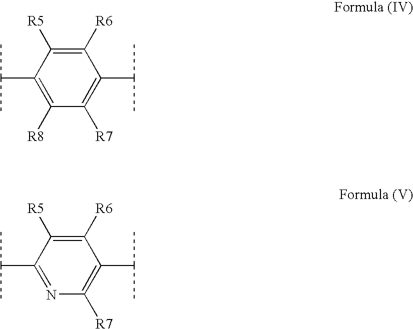
   W₃ is chosen from a hydrogen atom and C₁-C₆ alkyl radicals optionally substituted with at least one entity radical chosen from hydroxyl, alkoxy, amino, monoalkylamino, and dialkylamino radicals.
   W₅ is chosen from 5-membered nitrogenous heteroaromatic radicals linked to W₄ via the nitrogen atom of the ring of the said heteroaromatic radical, this heteroaromatic radical being chosen from pyrazolyl, pyrrolyl, imidazolyl, triazolyl and thiadiazolyl radicals, each of these heteroaromatic radicals optionally being substituted with at least one entity chosen from hydrogen, chlorine and fluorine atoms; C₁-C₆ alkyl radicals, optionally substituted with at least one radical chosen from hydroxyl, C₁-C₄ alkoxy, (poly)hydroxyalkoxy, amino, C₁-C₄ (di)alkylamino, C₂-C₄ (poly)hydroxyalkylamino, carboxyl, sulfonyl, alkoxycarbonyl and C₁-C₄ thioether radicals; phenyl radicals optionally substituted with at least one entity chosen from halogen atoms, and hydroxyl, C₁-C₂ alkoxy, amino, C₁-C₂ (di)alkylamino, carboxyl, sulfonyl, C₁-C₄ alkyl, and C₁-C₂ thioether radicals,
   R1, R2, R9 and R12, which may be identical or different, are chosen from optionally substituted phenyl radicals and C₁-C₈ alkyl radicals, optionally substituted with at least one radical chosen from hydroxyl, C₁-C₂ alkoxy, C₂-C₄ (poly)hydroxyalkoxy, amino and C₁-C₂ (di)alkylamino radicals,
   R5, R6, R7 and R8, which may be identical or different, are chosen from hydrogen atoms; chlorine atoms; bromine atoms; linear and branched C₁-C₈ hydrocarbon-based chains, which can form at least one 3- to 6-membered carbon-based ring, and which may be saturated or unsaturated, wherein at least one of the carbon atoms of the carbon-based chain may be replaced with an entity chosen from oxygen, nitrogen and sulfur atoms and SO₂ groups, and wherein the carbon atoms of which may be, independently of each other, substituted with at least one halogen atom; with the proviso that R5, R6, R7 and R8 do not comprise any peroxide bonds, or diazo or nitroso radicals.
   R3, R4, R10, R11, R13, R14 and R15, which may be identical or different, are chosen from hydrogen atoms, linear and branched C₁-C₁₆ hydrocarbon-based chains, which can form at least one 3- to 6-membered carbon-based ring, and which may be saturated or unsaturated, wherein at least one of the carbon atoms of the carbon-based chain may be replaced with an entity chosen from oxygen, nitrogen and sulfur atoms and SO₂ groups, and wherein the carbon atoms of which may be, independently of each other, substituted with at least one halogen atom; with the proviso that R3, R4, R10, R11, R13, R14 and R15 do not comprise any peroxide bonds, or diazo or nitroso radicals.

R4 with R13 and R14 with R15 may form a carbon-based aromatic ring, such as a phenyl.

The compounds of formula (XVII) are described, for example, in French Patent Application No. FR 2 844 269, the content of which is incorporated herein by reference.

Among the cationic azo or azomethine dyes that may be used as disclosed herein, non-limiting mention may also be made of Basic Blue 41, Basic Blue 67, Basic Brown 1, Basic Brown 4, Basic Brown 16, Basic Brown 17, Basic Red 18, Basic Red 22, Basic Red 46, Basic Red 76, Basic Red 104, Basic Red 118, Basic Violet 35, Basic Yellow 45, Basic Yellow 57 and Basic Yellow 67.

Among the cationic methine direct dyes that may be used as disclosed herein, non-limiting mention may be made of Basic Red 14, Basic Yellow 13 and Basic Yellow 29.

Further among the azo, methine and azomethine cationic direct dyes, non-limiting mention can be made of the compounds of formulae I, II, III and III′.

The at least one cationic direct dye of the present disclosure can be present in an amount ranging from 0.0005% to 15% by weight, such as from 0.005% to 10% by weight, relative to the total weight of the composition.

In the context of the present disclosure, the alcohol oxidase enzymes that may be used in the dye composition as disclosed herein belong to the category EC 1.1.3 of the enzyme nomenclature (see Enzyme Nomenclature, Academic Press Inc, 1992).

The enzymes may be chosen, for example, from primary alcohol oxidases (EC 1.1.3.13), secondary alcohol oxidases (EC 1.1.3.18), long hydrocarbon chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7), also known as aryl alcohol oxidases.

The alcohol oxidase enzymes form a category of 2-electron oxidoreductase enzymes.

The alcohol oxidase enzyme used in the dye composition according to the present disclosure may be derived from plant, animal and microorganism (bacterium, fungus, yeast, microalga or virus) extracts, from differentiated and undifferentiated cells, obtained in vivo and in vitro, which are genetically modified or unmodified, or synthetic (obtained via chemical or biotechnological synthesis).

Non-limiting examples that may be mentioned include enzymes extracted from the following species: Pinus, Gastropode, Manduca, Pichia, Candida, Pleurotus, Pseudomonas, Rhodococcus, Aspergillus, Kamagataella, Phanerochaete, Polyporus, Hansenula, Poria, Penicillium, and for further non-limiting example, the following species: Pinus strobus, which is a species of plant origin, Gastropode mollusc, Manduca sexta, which are of animal origin, Pichia sp. (pastoris, methanolica, angusta) and Candida sp. (boidinii, albicans, tropicalis), which are yeasts, Pleurotus pulmonarius Aspergillus niger, Kamagataella pastoris, Phanerochaete chrysosporium, Polyporus obtusus, Hansenula polymorpha, Poria contigua, Penicillium simplicissimum, which are fungi, and Pseudomonas pseudoalcaligenes, Rhodococcus erythropolis, which are bacteria.

For instance, the alcohol oxidase used may be derived from the strain Pichia pastoris.

For example, the at least one alcohol oxidase enzyme is present in the dye composition in an amount ranging from 0.05% to 20% by weight, relative to the total weight of the composition, for instance, from 0.1% to 10%, such as from 0.5% to 8% by weight, relative to the total weight of the dye composition.

The enzymatic activity of the at least one alcohol oxidase enzyme used in accordance with the present disclosure may be defined from the oxidation of the donor under aerobic conditions. The unit U is the amount of enzyme leading to the generation of 1 μmol of hydrogen peroxide per minute at a given pH and at a temperature of 25° C. For example, the at least one alcohol oxidase enzyme can be present in the dye composition in an amount ranging from 10³ U to 10⁵ U, such as from 2×10³ U to 5×10⁴ U, per 100 g of dye composition.

The at least one substrate for the enzyme is also known as a donor for the enzyme. The nature of this substrate varies as a function of the nature of the alcohol oxidase enzyme used. The at least one substrate for the enzyme in the compositions of the present disclosure can be, for example, an alcohol chosen from branched and unbranched, saturated and unsaturated, substituted and unsubstituted primary and secondary alcohols, long hydrocarbon chain alcohols and aromatic alcohols. For example, non-limiting mention may be made, among donors for the primary alcohol oxidases, of primary alcohols comprising from 1 to 6 carbon atoms; among donors for the aryl alcohol oxidases: benzyl alcohol, 4-tert-butylbenzyl alcohol, 3-hydroxy-4-methoxybenzyl alcohol, veratryl alcohol, 4-methoxybenzyl alcohol, cinnamyl alcohol; 2,4-hexadien-1-ol may also be used as donors for the aryl alcohol oxidases.

According to another aspect of the present disclosure, the at least one substrate for the enzyme can be a compound bearing at least one aliphatic or aromatic alcohol functional group, suitable for reaction with the enzyme used. The compound bearing at least one aliphatic or aromatic alcohol functional group may be, for example, an oxidation dye precursor or a cosmetically acceptable adjuvant, for example a polymer, a surfactant or a preserving agent bearing at least one alcohol functional group. For further example, the at least one substrate for the enzyme can be an oxidation dye precursor bearing at least one aliphatic or aromatic alcohol functional group. For example, N-(β-hydroxypropyl)-para-phenylenediamine, which bears a primary alcohol functional group, may serve as oxidation base and as a substrate for the alcohol oxidase. Similarly, oxidation couplers, such as meta- or para-aminophenol, may serve the two functions. Such precursors are described hereinbelow. In this aspect of the present disclosure, the use of other substrates for the enzyme is optional.

Thus, the present disclosure relates to a composition for dyeing keratin fibers, for instance human keratin fibers such as the hair, comprising, in a medium that is suitable for dyeing, at least one oxidation dye precursor; at least one alcohol oxidase enzyme; at least one substrate, bearing an alcohol functional group, for the enzyme, and at least one cationic direct dye chosen from azo, methine and azomethine cationic direct dyes, the substrate optionally being substituted (i.e. replaced) totally or partially with the oxidation dye precursor in the case where the oxidation dye precursor bears at least one functional group chosen from aliphatic and aromatic alcohol functional groups.

By using the composition in accordance with the present disclosure, the risks associated with the handling of hydrogen peroxide can be reduced. Furthermore, the concentration of preserving agents in the compositions according to the present disclosure may be reduced by providing compounds comprising at least one alcohol functional group that also have preserving properties.

For example, the at least one substrate can be present in an amount ranging from 0.01% to 60% by weight, relative to the total weight of the composition, such as from 0.05% to 30% by weight, relative to the total weight of the composition.

The standard oxidation bases may be chosen for instance, from para-phenylenediamines, bis(phenyl)alkylenediamines, para-aminophenols, ortho-aminophenols, heterocyclic bases, and the addition salts thereof.

Among the para-phenylenediamines that may be used, non-limiting mention may be made, by way of example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methyl-aniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-,N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-α-acetylaminoethyloxy-para-phenylenediamine, N-(β-methoxyethyl)-para-phenylenediamine, 4-aminophenylpyrrolidine, 2-thienyl-para-phenylenediamine, 2-α-hydroxyethylamino-5-aminotoluene, and the acid addition salts thereof.

Among the para-phenylenediamines mentioned above, further non-limiting mention can be made of para-phenylenediamine, para-tolylenediamine, 2-isopropyl-para-phenylenediamine, 2-βhydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine and the acid addition salts thereof.

Among the bis(phenyl)alkylenediamines, non-limiting mention may be made, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethyleneiamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and the acid addition salts thereof.

Among the para-aminophenols, mention may be made, by way of non-limiting example, of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and the acid addition salts thereof.

Among the ortho-aminophenols, mention may be made, by way of non-limiting example, of 2-aminophenol, 2-amino-5-methylphenol, 2-amino-6-methylphenol, 5-acetamido-2-aminophenol, and the acid addition salts thereof.

Among the heterocyclic bases, mention may be made, by way of non-limiting example, of pyridine derivatives, pyrimidine derivatives and pyrazole derivatives.

Among the pyridine derivatives, further non-limiting mention may be made of the compounds described, for example, in the British Patent Nos. GB 1 026 978 and GB 1 153 196, as well as 2,5-diaminopyridine, 2-(4-methoxyphenyl)amino-3-aminopyridine, 3,4-diaminopyridine, and the addition salts thereof with an acid. Other pyridine oxidation bases that are useful in the present disclosure, include, by way of non-limiting example, the 3-aminopyrazolo[1,5-a]pyridine oxidation bases and the addition salts thereof described, for example, in French Patent Application No. FR 2 801 308. Non-limiting examples that may be mentioned include pyrazolo[1,5-a]pyrid-3-ylamine; 2-acetylaminopyrazolo-[1,5-a]pyrid-3-ylamine; 2-morpholin-4-ylpyrazolo[1,5-a]pyrid-3-ylamine; 3-aminopyrazolo[1,5-a]pyrid-2-carboxylic acid; 2-methoxypyrazolo[1,5-a]pyrid-3-ylamino; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-yl-pyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]ethanol; 2-[(3-aminopyrazolo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]pyrid-7-ol; and also the addition salts thereof with an acid.

Among the pyrimidine derivatives, non-limiting mention may be made of the compounds described, for example, in German Patent No. DE 2 359 399; Japanese Patent Nos. JP 88-169 571 and JP 05 163 124; European Patent No. EP 0 770 375 or International Patent Application No. WO 96/15765, such as 2-hydroxy-4,5,6-triaminopyrimidine, 2,4-dihydroxy-5,6-diaminopyrimidine and pyrazolopyrimidine derivatives such as those mentioned in French Patent Application No. FR-A-2 750 048, and among which non-limiting mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine, 3-amino-5-methyl-7-imidazolylpropylaminopyrazolo[1,5-a]pyrimidine, and the acid addition salts thereof, and the tautomeric forms thereof, when a tautomeric equilibrium exists.

Among the pyrazole derivatives, non-limiting mention may be made of the compounds described in German Patent Nos. DE 3 843 892 and DE 4 133 957, International Patent Application Nos. WO 94/08969 and WO 94/08970, French Patent Application No. FR-A-2 733 749 and German Patent Application No. DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropyl pyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the acid addition salts thereof.

For example, the at least one oxidation base can be present in an amount ranging from 0.0001% to 20%, such as from 0.005% to 6% by weight, relative to the total weight of the composition.

Among standard oxidation couplers that may be used, for example, non-limiting mention may be made of meta-phenylenediamines, meta-aminophenols, meta-diphenols, naphthalene-based couplers, heterocyclic couplers, and also the addition salts thereof.

Non-limiting examples that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, and the addition salts thereof.

For example, the at least one oxidation coupler can be present in an amount ranging from 0.0001% to 20%, such as from 0.005% to 6% by weight, relative to the total weight of the composition.

The acid-addition salts that may be used as disclosed herein for the oxidation bases and couplers can be chosen, for instance, from the hydrochlorides, hydrobromides, sulfates, citrates, succinates, tartrates, lactates, tosylates, benzenesulfonates, phosphates and acetates. The base-addition salts that may be used in the context of the present disclosure can be chosen, for example, from the addition salts with sodium hydroxide, potassium hydroxide, ammonia, amines and alkanolamines.

The dye composition in accordance with the present disclosure may further comprise at least one adjuvant conventionally used in hair dye compositions, such as antioxidants, penetrating agents, sequestering agents, fragrances, buffers, dispersants, surfactants, thickening polymers, conditioning agents, for instance volatile or non-volatile, modified or unmodified silicones, cationic polymers, cations, film-forming agents, ceramides, preserving agents, opacifiers, vitamins or provitamins, oxidation dyes, and direct dyes other than azo, methine or azomethine cationic direct dyes.

The at least one adjuvant can be present in an individual amount ranging from 0.01% to 20% by weight, relative to the weight of the composition.

Needless to say, a person skilled in the art will take care to select this or these optional additional compound(s) such that the beneficial properties intrinsically associated with the oxidation dye composition in accordance with the present disclosure are not, or are not substantially, adversely affected by the envisaged addition(s).

The medium that is suitable for dyeing, also known as the dye support, can consists of water or comprises a mixture of water and at least one organic solvent to dissolve the compounds that would not be sufficiently water-soluble. The at least one solvent may, where appropriate, be an enzyme substrate such as ethanol and isopropanol. This may also be an enzyme nonsubstrate such as polyol ethers, for instance 2-butoxyethanol, propylene glycol, propylene glycol monomethyl ether, diethylene glycol monomethyl ether, monoethyl ether, phenoxyethanol, and mixtures thereof.

The at least one solvent may be present in an amount, for example, ranging from 1% to 40% by weight, relative to the total weight of the dye composition, such as from 5% to 30% by weight.

The pH of the dye composition in accordance with the present disclosure can range from 6 and 11, for instance, such as from 7 to 10. It may be adjusted for the desired value using acidifying or basifying agents usually used in the dyeing of keratin fibers, or alternatively using standard buffer systems.

Among the acidifying agents that may be used, non-limiting mention can be made of, for example, mineral or organic acids, for instance hydrochloric acid, orthophosphoric acid, sulfuric acid, carboxylic acids, for instance acetic acid, tartaric acid, citric acid and lactic acid, and sulfonic acids.

Among the basifying agents that may be used, non-limiting mention may be made of, for example, aqueous ammonia, alkyl metal carbonates, alkanolamines such as monoethanolamine, diethanolamine and triethanolamine and also derivatives thereof, sodium hydroxide, potassium hydroxide and the compounds of formula (III): 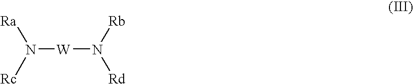
wherein W is chosen from propylene residues optionally substituted with a hydroxyl group or a C₁-C₄ alkyl radical; Ra, Rb, Rc and Rd, which may be identical or different, are chosen from hydrogen atoms, C₁-C₄ alkyl and C₁-C₄ hydroxyalkyl radicals.

The dye composition may be in various forms, such as in the form of liquids, creams or gels, or in any other form that is suitable for dyeing keratin fibers, such as human hair.

When the at least one oxidation dye and the at least one alcohol oxidase are present in the same ready-to-use composition, the said composition is, in some embodiments, free of oxygen gas, so as to avoid any premature oxidation of the at least one oxidation dye.

Another aspect of the present disclosure is a process for dyeing keratin fibers, for instance human keratin fibers such as the hair, such that at least one dye composition according to the present disclosure is applied to the fibers, the duration of this application being a period of time that is sufficient to develop a desired coloration. On contact with atmospheric oxygen, the color is revealed by bringing together the at least one alcohol oxidase enzyme and its at least one substrate.

In one embodiment of the present disclosure, the composition is applied to the keratin fibers, and after leaving it to act for a period of time ranging from 3 to 60 minutes, such as from 5 to 40 minutes, the keratin fibers are rinsed, washed with shampoo, rinsed again and then dried.

In another aspect of the present disclosure, when the dye composition is a composition in ready-to-use form, it comprises, in a medium that is suitable for dyeing keratin fibers, at least one oxidation dye precursor, at least one alcohol oxidase enzyme, at least one substrate for the enzyme, and at least one cationic direct dye chosen from azo, methine and azomethine cationic direct dyes, and the mixture is then stored in anaerobic form, free of oxygen gas.

According to another aspect of the present disclosure, the dyeing process includes separately storing, on the one hand, a composition (A) comprising in a medium that is suitable for dyeing keratin fibers, at least one oxidation dye precursor, and, on the other hand, a composition (B) comprising, in a medium that is suitable for dyeing keratin fibers, at least one alcohol oxidase enzyme, wherein the composition (A) and/or the composition (B) comprise at least one substrate for the said enzyme, and the composition (A) and/or the composition (B) comprise at least one cationic direct dye chosen from azo, methine and azomethine cationic direct dyes, and the compositions (A) and (B) are mixed together at the time of use before applying this mixture to the keratin fibers. This process can also comprise, for example, separately storing, on the one hand, a composition (A) comprising, in a medium that is suitable for dyeing keratin fibers, at least one oxidation dye precursor, at least one substrate for the alcohol oxidase enzyme and at least one cationic direct dye chosen from azo, methine and azomethine cationic direct dyes and, on the other hand, a composition (B) comprising, in a medium that is suitable for dyeing keratin fibers, at least one alcohol oxidase enzyme, wherein the compositions (A) and (B) are mixed together at the time of use, before applying this mixture to the keratin fibers.

The color may be developed at acidic, neutral or alkaline pH. In the case where the process is performed using a composition (A) comprising at least one oxidation dye precursor, at least one substrate for the enzyme and at least one cationic direct dye chosen from azo, methine and azomethine cationic direct dyes, and a composition (B) comprising at least one alcohol oxidase enzyme, the enzyme may be added to the composition as disclosed herein just at the time of use, or may be used starting with a composition comprising it, which is applied simultaneously with or sequentially to the composition of the present disclosure.

The composition (B), also referred to as the oxidizing composition, may also comprise at least one adjuvant conventionally used in hair dye compositions and as defined above.

The pH of the “oxidizing” composition (B) is such that, after mixing with the “dye” composition (A), the pH of the resulting composition applied to the keratin fibers can range, for example, from 6 to 11, such as from 7 to 10. It may be adjusted to the desired value using acidifying or basifying agents usually used in the dyeing of keratin fibers and as defined above.

For example, the application of the composition according to the present disclosure can be performed at a temperature ranging from room temperature to 220° C., for instance, from room temperature to 60° C.

Another aspect of the present disclosure is a multi-compartment device or dyeing “kit”, wherein at least one first compartment comprises the composition (A) as defined above, and at least one second compartment comprises the composition (B) as defined above. This kit may optionally be equipped with devices for applying the desired mixture to the hair, such as the devices described in French Patent No. FR 2 586 913.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements. The following example is intended to illustrate the invention in a non-limiting manner.

### EXAMPLE

The dye composition below was prepared in accordance with the present disclosure.

| Constituent | Amount |
|---|---|
| Acid Black 1 (azo, methine or azomethine | 0.4 g |
| cationic direct dye) | |
| Ethanol (donor substrate) | 25 g |
| para-Phenylenediamine (dye precursor) | 3 × 10⁻³ mol |
| meta-Aminophenol (coupler) | 3 × 10⁻³ mol |
| Alcohol oxidase | 20 000 units U |
| 2-Amino-2-methyl-1-propanol | qs pH 7 |
| Distilled water | qs 100 g |

The alcohol oxidase used is the product sold by the company Biozyme Laboratories in liquid form at a concentration of 1980 units/ml.

The unit U is the amount of enzyme leading to the generation of 1 μmol of hydrogen peroxide per minute at pH 7.5 (100 mM phosphate buffer) and at a temperature of 25° C.

The above compositions were applied to locks of natural and permanent-waved grey hair comprising 90% white hairs, and were left to act for 30 minutes. The bath ratio was set at 5. The alcohol oxidase was added extemporaneously. The hair was then rinsed, washed with a standard shampoo and then dried.

1. A composition for dyeing keratin fibers, comprising, in a medium that is suitable for dyeing, at least one oxidation dye precursor; at least one alcohol oxidase enzyme; at least one substrate for the enzyme, wherein the substrate bears an alcohol functional group; and at least one cationic direct dye chosen from azo, methine and azomethine cationic direct dyes, wherein when the at least one oxidation dye precursor bears at least one functional group chosen from aliphatic and aromatic alcohol functional groups, the at least one substrate is optionally totally or partially substituted with the at least one oxidation dye precursor.

2. The composition according to claim 1, wherein the at least one cationic direct dye chosen from azo, methine and azomethine cationic direct dyes is chosen from:
the compounds of formula (I): 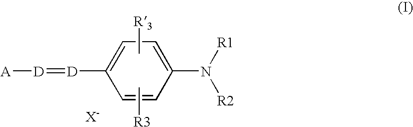
wherein D is chosen from a nitrogen atom and CH groups,
R1 and R2, which may be identical or different, are chosen from hydrogen atoms, alkyl radicals C₁-C₄ optionally substituted with a radical chosen from CN, OH and NH₂ radicals, or form, with a carbon atom of the benzene ring, a nitrogen heterocycle optionally also comprising an oxygen atom or a nitrogen atom, which may be substituted with at least one alkyl radical C₁-C₄; and R₁ and R₂ may form a 4′-aminophenyl radical;
R3 and R′₃, which may be identical or different, are chosen from hydrogen atoms, halogen atoms chosen from chlorine, bromine, iodine and fluorine, and cyano, alkoxy C₁-C₄ and acetyloxy radicals,
X⁻ is an anion,
A is a radical chosen from those of A1 to A18: 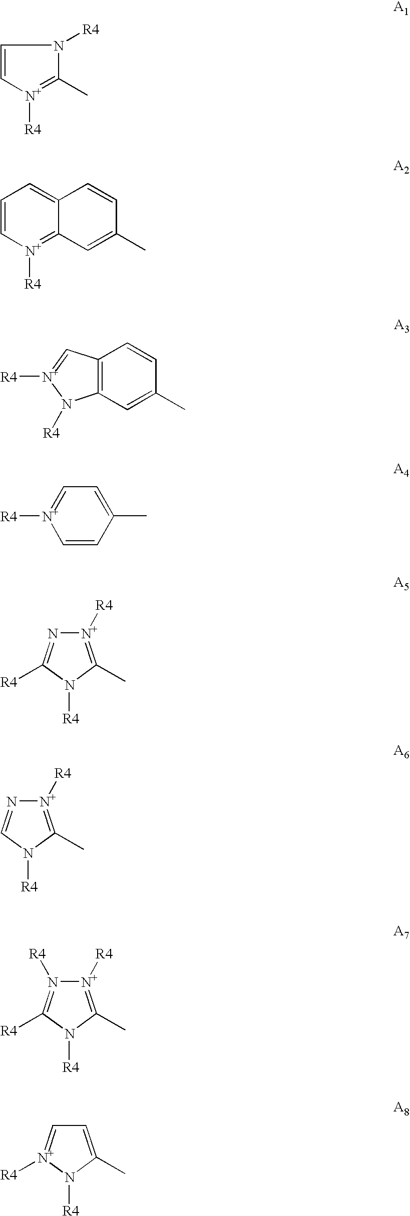 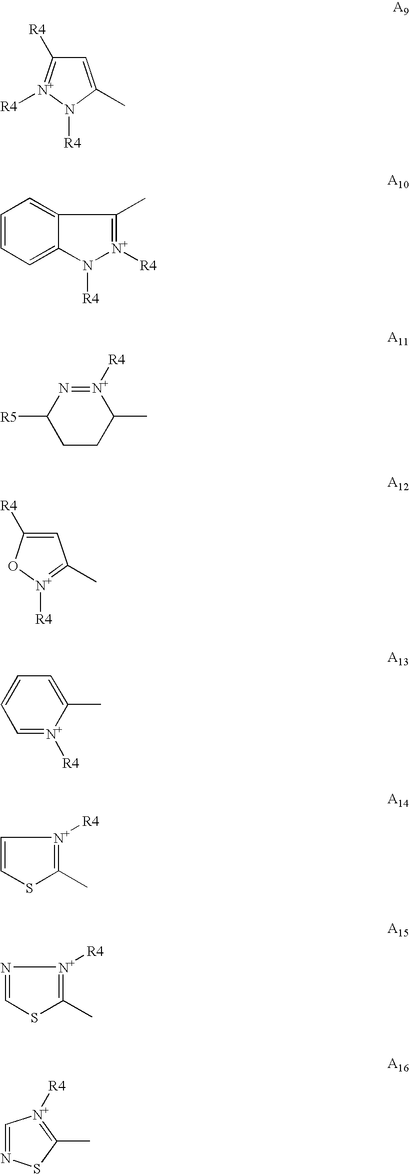 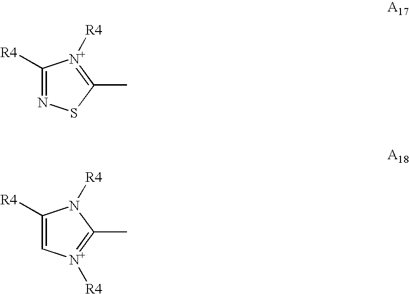
R₄ is chosen from C₁-C₄ alkyl radicals optionally substituted with a hydroxyl radical and R₅ is chosen from C₁-C₄ alkoxy radicals;
the compounds of formula (II): 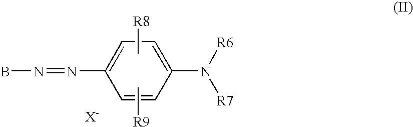
wherein R6 is chosen from a hydrogen atom and C₁-C₄ alkyl radicals;
R7 is chosen from a hydrogen atom; alkyl radicals optionally substituted with a CN radical or with an amino group; 4′-aminophenyl radicals; or forms with R6 a nitrogen heterocycle possibly also comprising an oxygen atom and/or a nitrogen atom which may be substituted with a C₁-C₄ radical;
R₈ and R₉, which may be identical or different, are chosen from hydrogen atoms, halogen atoms, and C₁-C₄ alkyl, C₁-C₄ alkoxy, and CN radicals;
X⁻ is an anion;
B is a radical chosen from those of B₁ to B₆: 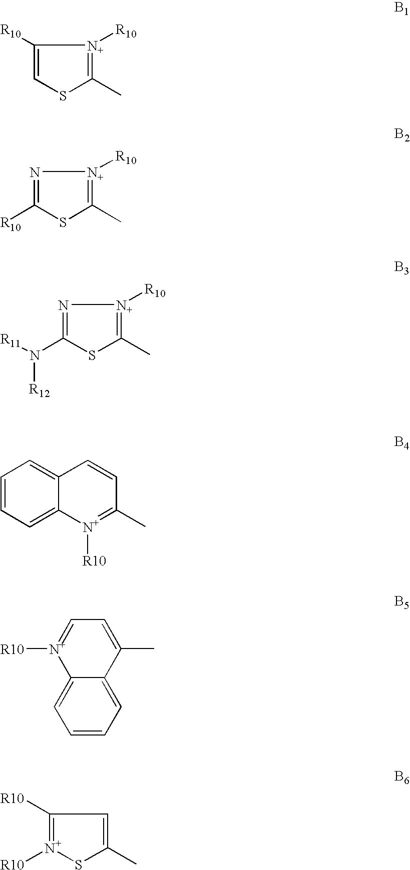
wherein R10 is chosen from C₁-C₄ alkyl radicals;
R11 and R12, which may be identical or different, are chosen from hydrogen atoms and C₁-C₄ alkyl radicals;
the compounds of formulae (III) and (III′): 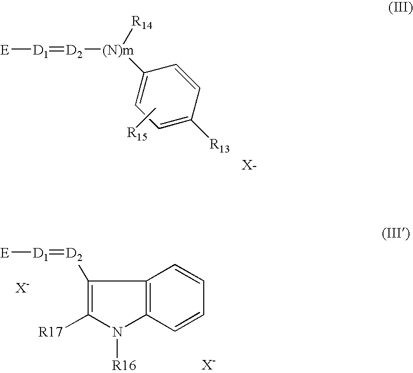
wherein R₁₃ is chosen from a hydrogen atom, C₁-C₄ alkoxy radicals, halogen atoms, and amino radicals;
R₁₄ is chosen from a hydrogen atom, and C₁-C₄ alkyl radicals, or forms, with a carbon atom of the benzene ring, a heterocycle optionally comprising oxygen and/or nitrogen, substituted with at least one C₁-C₄ group;
R₁₅ is chosen from a hydrogen atom and halogen atoms;
R16 and R17, which may be identical or different, are chosen from hydrogen atoms and C₁-C₄ alkyl radicals;
D₁ and D₂, which may be identical or different, are chosen from nitrogen atoms and CH groups;
m is equal to 0 or 1;
it being understood that when R₁₃ is an unsubstituted amino group, then D₁ and D₂ are both simultaneously a —CH group and m is equal to 0;
X⁻ is an anion;
E is a radical chosen from those of E₁ to E₈: 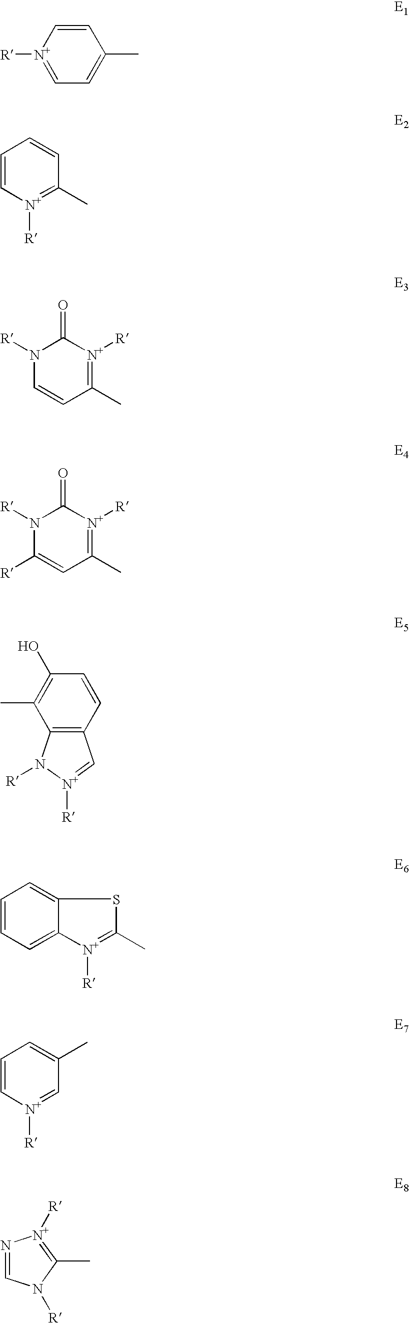
wherein R′ is chosen from C₁-C₄ alkyl radicals,
when m is 0 and D₁ is a nitrogen atom, then E may also be chosen from the radicals of E₉: 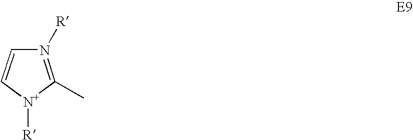
wherein R′ is chosen from C₁-C₄ alkyl radicals;
the compounds of formula (IV):
W₁—W₂—N═N—W₃-L-W₄—N═N—W₅—W₆ (IV)
wherein W₁ and W₆, which may be identical or different, are chosen from NR′₁R′₂ radicals;
W₂ and W₅, which may be identical or different, are chosen from carbon-based, pyridyl and pyridazinyl aromatic groups of formula (IVa): 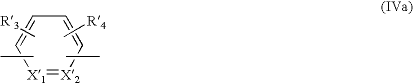
W₃ and W₄, which may be identical or different, are chosen from heteroaromatic radicals of formulae (IVb) and (IVc): 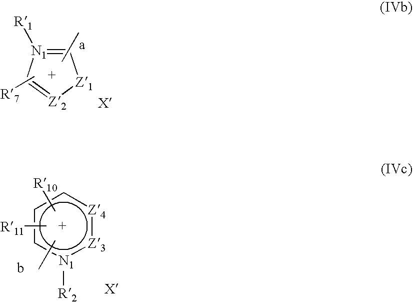
wherein X′₁ is chosen from a nitrogen atom and CR₁₅ radicals,
X′₂ is chosen from a nitrogen atom and CR₁₆ radicals
Z′₁ is chosen from oxygen and sulfur atoms, and NR₁₈ radicals
Z′₂ is chosen from a nitrogen atom and CR′₉ radicals
Z′₃ is chosen from a nitrogen atom and CR′₁₂ radicals
Z′₄ is chosen from a nitrogen atom and CR′₁₃ radicals
N₁ of the 5-membered ring of formula (IVb) is linked to the group L of formula (IV) via the group R′₁ and the bond a of the same 5-membered ring is linked to the azo group of formula (IV);
the bond b of the 6-membered ring of formula (IVc) is linked to the azo group of formula (IV) and N₁ of the 6-membered ring of formula (IVc) is linked to the group L of formula (IV) via the group R₁₂;
L, R′₁, R′₁₂, R′₃, R′₄, R′₅, R′₆, R′₇, R′₉, R′₁₀, R′₁₁, R′₁₂ and R′₁₃, which may be identical or different, are chosen from linear and branched C₁-C₁₆ hydrocarbon-based chains that can form at least one 3- to 6-membered carbon-based ring and which may be saturated or unsaturated, wherein at least one of the carbons of the carbon-based chain may be replaced with an entity chosen from oxygen, nitrogen and sulfur atoms and SO₂ groups, and the carbon atoms of which may be, independently of each other, substituted with at least one halogen atom;
R′₁, R′₂, R′₃, R′₄, R′₅, R′₆, R′₇, R′₉, R′₁₀, R′₁₁, R′₁₂ and R′₁₃ may also be chosen from hydrogen atoms; with the proviso that L, R′₁, R′₂, R′₃, R′₄, R′₅, R′₆, R′₇, R′₉, R′₁₀, R′₁₁, R′₁₂ and R′₁₃ do not comprise any peroxide bonds or diazo or nitroso radicals and L is a divalent radical;
R′₈ is chosen from linear and branched C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl, and sulfonic radicals; and optionally substituted phenyl radicals;
R′₇ with R′₉, R′₁₀ with R′₁₁, R′₁₂ with R′₁₃ may also form a carbon-based aromatic ring such as a phenyl;
X′ is chosen from organic and mineral anions;
the compounds of formula (V):
W₇—N═N—W₈—N═N—W₉ (V)
wherein:
W₇ and W₉, which may be identical or different, are chosen from heteroaromatic radicals of formulae Va and Vb: 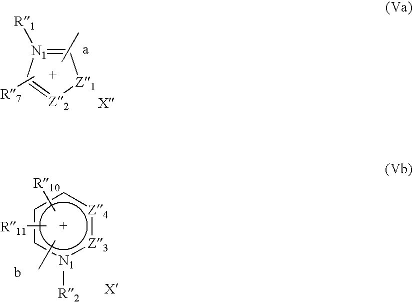
wherein
W₈ is chosen from carbon-based, pyridyl and pyridazinyl aromatic groups of formula (Vc): 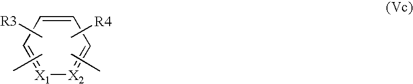
in which:
X₁ is chosen from a nitrogen atom and CR₁₅ radicals
X₂ is chosen from a nitrogen atom and CR₁₆ radicals
Z″₁ is chosen from oxygen and sulfur atoms, and NR″₈ radicals
Z″₂ is chosen from a nitrogen atom and CR″₉ radicals,
Z″₃ is chosen from a nitrogen atom and radical CR″₁₂ radicals
Z″₄ is chosen from a nitrogen atom and CR″₁₃ radicals the bond a of the 5-membered cationic ring of formula (Va) is linked to the azo group of formula (V);
the bond b of the 6-membered cationic ring of formula (Vb) is linked to the azo group of formula (V);
R₃, R₄, R″₁, R′₂, R″₅, R″₆, R″₇, R″₈, R″₉, R″₁₀, R″₁₁, R″₁₂ and R″₁₃, which may be identical or different, are chosen from hydrogen atoms, linear and branched, saturated and unsaturated C₁-C₁₆ hydrocarbon-based chains, which can form at least one 3- to 6-membered carbon-based ring, wherein at least one of the carbon atoms of the carbon-based chain may be replaced with an entity chosen from oxygen, nitrogen and sulfur atoms and SO₂ groups, and the carbon atoms of which may be, independently of each other, substituted with at least one halogen atom;
with the proviso that R₃, R₄, R″₁, R″₂, R″₅, R″₆, R″₇, R″₈, R″₉, R″₁₀, R″₁₁, R″₁₂ and R″₁₃ do not comprise any peroxide bonds or diazo or nitroso radicals;
R″₇ and R″₉, R″₁₀ and R″₁₁, R″₁₂ and R″₁₃ may also form a carbon-based ring;
X′ and X″ are chosen from organic and mineral anions;
the compounds of formula (VIa) and (VIb): 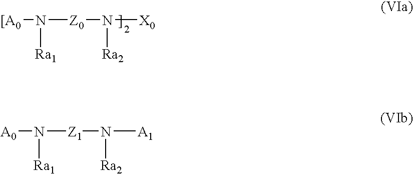
wherein A₀ and A₁, which may be identical or different, are chosen from radicals of formula (VId): 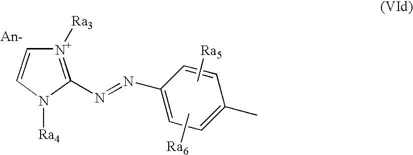
wherein
Z₀ is chosen from aliphatic radicals
Z₁ is chosen from alkyl radicals
Rₐ₁ and Rₐ₂, which may be identical or different, are chosen from hydrogen atoms, (C₁-C₄)alkyl radicals, and (C₁-C₄)alkyl radicals substituted with at least one halogen atom, and hydroxyl, carboxyl, cyano, (C₁-C₄)alkoxy, and (C₁-C₄)alkoxy radicals substituted with at least one radical chosen from hydroxyl, (C₁-C₄)alkoxy, amino, alkylamino, dialkylamino, aminocarbonyl, phenyl, phenoxy and phenylaminocarbonyl radicals, in which the phenyl radical is unsubstituted or substituted with a (C₁-C₄)alkyl, (C₁-C₄)alkoxy or phenoxy radical;
Rₐ₁ and Rₐ₂ may also form, together with the two nitrogen atoms that bear them and the radical Z₀, a piperazine radical;
X₀ is a bridging radical chosen from —CO—, —COCH₂—, —CH₂CO—, —COCO—, 1,4-dicarbonylphenyl and —CH₂—CH₂— radicals, or a triazine chosen from the formulae: 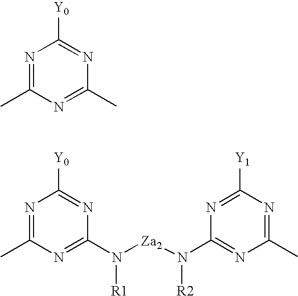
wherein Y₀ and Y₁, which may be identical or different, are chosen from halogen atoms, and hydroxyl, amino, monoalkylamino, dialkylamino, 1-piperidino, morpholino, and 1-piperazino radicals, the piperazino radical being unsubstituted or substituted on the nitrogen atom not attached to the triazine ring with a (C₁-C₄)alkyl radical, wherein the alkyl radicals are unsubstituted or substituted with hydroxyl, amino, mono(C₁-C₄)alkylamino or di(C₁-C₄)alkylamino radicals;
R₁ and R₂ are chosen from hydrogen atoms and C₁-C₄ alkyl radicals optionally substituted with a hydroxy radical;
Zₐ₂ is chosen from C₂-C₈ alkylene radicals, or forms, with the two adjacent nitrogen atoms and the radicals R₁ and R₂, a piperazine ring;
Rₐ₃ and Rₐ₄, which may be identical or different, are chosen from hydrogen atoms; (C₁-C₄)alkyl radicals; C₁-C₄ alkyl radicals substituted with at least one entity chosen from halogen atom, and hydroxyl, carboxyl and cyano radicals; (C₁-C₄)alkoxy radicals; (C₁-C₄)alkoxy radicals substituted with a hydroxyl or (C₁-C₄)alkoxy radical; and amino, alkylamino, dialkylamino, aminocarbonyl, phenyl, phenoxy and phenylaminocarbonyl radicals, in which the phenyl radical is unsubstituted or substituted with a (C₁-C₄)alkyl, (C₁-C₄)alkoxy or phenoxy radical;
Rₐ₅ and Rₐ₆, which may be identical or different, are chosen from hydrogen atoms, C₁-C₄ alkyl radicals, C₁-C₄ alkoxy radicals, optionally substituted with a hydroxyl, carboxyl, halogen, cyano, (C₁-C₄)alkoxy optionally substituted with a hydroxyl or (C₁-C₄)alkoxy radical; and amino, alkylamino, dialkylamino, aminocarbonyl, phenyl, phenoxy and phenylaminocarbonyl radicals, in which the phenyl radical is unsubstituted or substituted with a (C₁-C₄)alkyl, (C₁-C₄)alkoxy or phenoxy radical,
An⁻ is an anion;
the compounds of formula (VIc): 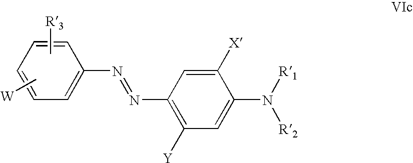
wherein the cationic charge number is 2;
X′ and Y, which may be identical or different, are chosen from hydrogen atoms, halogen atoms, and (C₁-C₄)alkyl, (C₁-C₄)alkoxy, (C₁-C₄)alkylcarbonylamino, arylcarbonylamino, ureido and arylureido radicals;
R′₁ is chosen from a hydrogen atom, substituted alkyl and aryl radicals, unsubstituted alkyl and aryl radicals, and R′₂ radicals;
R′₂ is chosen from radicals of formula: 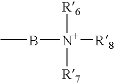
wherein B is chosen from linear and branched alkylene radicals;
R′₆ is chosen from a hydrogen atom and substituted and unsubstituted alkyl radicals;
R′₇ and R′₈, which may be identical or different, are chosen from substituted and unsubstituted alkyl radicals;
R′₆ and R′₇ together with the nitrogen atom, may also form a substituted or unsubstituted 5-, 6- or 7-membered ring, which may comprise other hetero atoms, or alternatively R₁₆ and R′₇ and R′₈ together can form a pyridinium ring;
R′₃ is chosen from hydrogen and halogen atoms, and (C₁-C₄)alkyl and (C₁-C₄)alkoxy radicals;
W is a radical of formula: 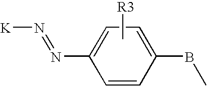
wherein K is a coupling compound chosen from those of formula (f), (g) or (h): 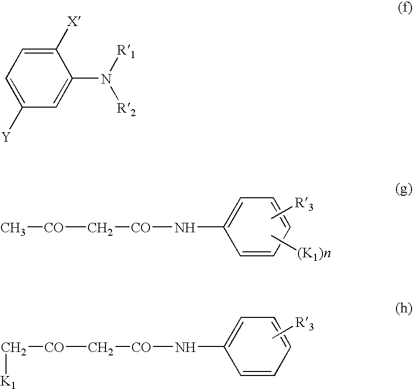
wherein,
X′ and Y, which may be identical or different, are chosen from hydrogen atoms, halogen atoms, and C₁-C₄ alkyl, C₁-C₄ alkoxy, (C₁-C₄)alkylcarbonylamino, arylcarbonylamino, ureido and arylureido radicals,
R′₁ is chosen from a hydrogen atom, substituted alkyl radicals, substituted aryl radicals, unsubstituted alkyl radicals, unsubstituted aryl radicals, and R′₂ radicals,
R′₂ is a radical of formula: 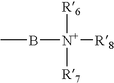
wherein B is chosen from linear and branched alkylene radicals;
R′₆ is chosen from a hydrogen atom and substituted and unsubstituted alkyl radicals;
R′₇ and R′₈, which may be identical or different, are chosen from substituted and unsubstituted alkyl radicals;
R′₁₆ and R′₇ together with the nitrogen atom, can also form a substituted or unsubstituted 5-, 6- or 7-membered ring, which may comprise other hetero atoms, or alternatively R′₆ and R′₇ and R′₈ together can form a pyridinium ring;
R′₃ is chosen from hydrogen and halogen atoms, and (C₁-C₄)alkyl and (C₁-C₄)alkoxy radicals;
n is equal to 1 or 2,
K₁ is chosen from radicals of formula —N⁺R′₆R′₇R′₈;
R3 has the same meanings as Rₐ₃,
B is a bridging radical chosen from the radicals: 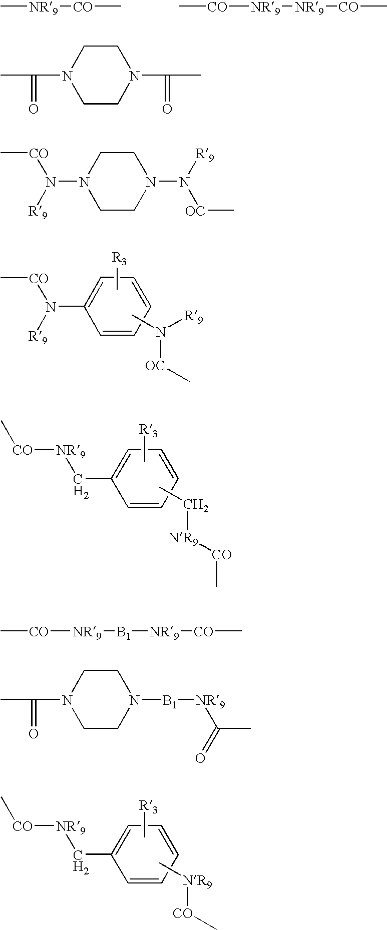
wherein R′₉ is chosen from a hydrogen atom, and C₁-C₂ alkylene radicals; B₁ is chosen from substituted and unsubstituted (C₂-C₄)alkylene radicals, the alkylene radical being linear or branched and possibly being interrupted with at least one entity chosen from —NR′₉— radicals, and —O— and —S— atoms;
the compounds of formulae (VIIIa) and (VIIIb):
Z₁-N═N-A₁-(A₃)ₙ-Z₂ (VIIIa) Z₁-N═N-A₂ (VIIIb)
wherein n is equal to 0 or 1,
Z₁ is chosen from 5- and 6-membered cationic heteroaromatic radicals of the formulae: 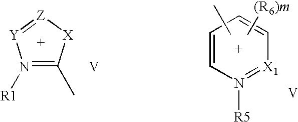
wherein X is chosen from NR₃ radicals, and S, and O atoms, Z is chosen from CR₂ radicals and N atoms, and Y is chosen from CR₄ radicals and N atoms, with the following conditions:
when X is chosen from NR₃ radicals and an oxygen atom, and Z is chosen from CR₂ radicals, then Y is chosen from CR₄ radicals and a nitrogen atom,
when X is a sulfur atom, then Z is a nitrogen atom or Y is a nitrogen atom,
when X is a sulfur atom and Z is a nitrogen atom, then Y is chosen from CR₄ radicals,
X₁ is chosen from CR₆ radicals and a nitrogen atom,
m is an integer equal to 0, 1, 2 or 3,
R₁, R₃, R₅, which may be identical or different, are chosen from saturated and unsaturated, linear and branched C₁-C₁₀ hydrocarbon-based chains which can form an optionally aromatic 5- to 7-membered carbon-based ring, wherein at least one of the carbon atoms of the chain may be replaced with an entity chosen from oxygen, nitrogen, halogen and sulfur atoms, and SO₂ groups, with the exception of the carbon linked to the nitrogen atom of the two preceding rings, and with the proviso that the radicals R₁ and R₃ or R₅ do not comprise any peroxide bonds, or diazo, nitro or nitroso radicals, R₂, R₄ and R₆, which may be identical or different, are chosen from hydrogen atoms, saturated and unsaturated, linear and branched C₁-C₁₀ hydrocarbon-based chains that can form an optionally aromatic 5- to 7-membered carbon-based ring; wherein at least one of the carbon atoms may be replaced with at least one entity chosen from oxygen, nitrogen and sulfur atoms and SO₂ groups, with the proviso that the radicals R₂, R₄ or R₆ do not comprise any peroxide bonds, or diazo, nitro or nitroso radicals, the radicals R₂ and R₄ together possibly forming an aromatic carbon-based ring,
V is chosen from organic and mineral anions,
A₁ and A₃, which may be identical or different, are divalent radicals chosen from thos of formulae (VIIIc) and (VIIId): 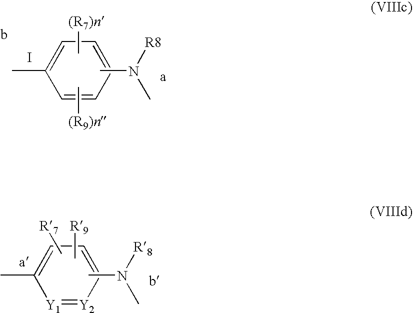
wherein
n′ is an integer equal to 0, 1, 2 or 3.
n″ is an integer equal to 0 or 1.
Y₁═Y₂ is chosen from C═N and N═N;
when n is 0, then either the bond a of the group A₁ of formula (VIIIc) is linked to the function Z₂ of formula (VIIIa), or the bond b′ of the group A₁ of formula (VIIId) is linked to the function Z₂ of formula (VIIIa)
when n is 1, the compounds of formula (VIIIa) are chosen from the four formulae below:
Z₁-N═N-(VIIIc)-(VIIId)-Z₂
Z₁-N═N-(VIIIC)-(VIIIC)-Z₂
Z₁-N═N-(VIIId)-(VIIIc)-Z₂
Z₁-N═N-(VIIId)-(VIIId)-Z₂
wherein the divalent radicals (VIIIc) and (VIIId) are linked together or to Z₂ or to the nitrogen atom of the azo group via the linkers a, a′, b and b′;
R₈ and R′₁₈, which may be identical or different, are non-cationic groups chosen from hydrogen atoms, linear and branched C₁-C₁₀ hydrocarbon-based chains that can form an optionally aromatic 5- to 7-membered carbon-based ring, wherein at least one of the carbon atoms of the hydrocarbon-based chain possibly being replaced with at least one entity chosen from oxygen, nitrogen and sulfur atoms and SO₂ groups, with the exception of the carbon linked to the nitrogen atom, and with the proviso that the radicals R₈ or R₁₈ do not comprise any peroxide bonds, or diazo, nitro or nitroso radicals;
R₇, R₉, R′₇ and R′₉, which may be identical or different, are chosen from non-cationic groups chosen from hydrogen atoms, saturated and unsaturated, linear and branched C₁-C₁₀ hydrocarbon-based chains that can form an optionally aromatic 5- to 7-membered carbon-based ring; wherein at least one of the carbon atoms may be replaced with at least one entity chosen from oxygen, nitrogen and sulfur atoms and SO₂ groups, with the proviso that the radicals do not comprise any peroxide bonds, or diazo, nitro or nitroso radicals, as defined for R₂, and cationic groups Z₃, on condition that only one of the groups R₇, R₉, R′₇ and R′₉ is cationic;
R₇ with R₈ or, respectively, R′₇ with R′₈ can together form a saturated 5- or 6-membered heterocycle;
Z₃ is a cationic group chosen from those of formula (VIIIe):
—(B)_{n′″}-D (VIIIe)
wherein:
B is chosen from linear and branched hydrocarbon-based chains comprising from 1 to 15 carbon atoms, which can form at least one optionally aromatic 3- to 7-membered ring, and at least one carbon atom of which may be replaced with an entity chosen from oxygen, nitrogen and sulfur atoms and SO₂ radicals, with the exception of the carbon linked to the nitrogen atom, with the proviso that B does not comprise any peroxide bonds, or any diazo, nitro or nitroso radicals;
the radical B is linked to D via any of the atoms of the radical D;
n″′ is 0 or 1;
D is chosen from the cationic groups of formulae (VIIIf) and (VIIIg): 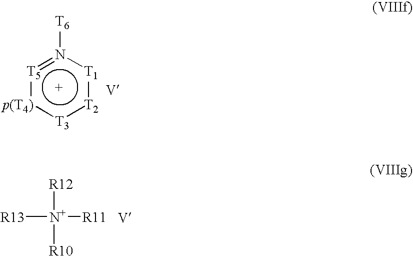
wherein:
p is 0 or 1;
T₁, T₂, T₃ and T₄, which may be identical or different, are chosen from oxygen atoms, sulfur atoms, nitrogen atoms which are unsubstituted or substituted with a radical R₁₄, and carbon atoms which are unsubstituted or substituted with one or two radicals R₁₄, which may be identical or different;
T₅ is chosen from a nitrogen atom or a carbon atom that is unsubstituted or substituted with a radical R₁₄;
T₆ is chosen from linear and branched, optionally aromatic hydrocarbon-based chains comprising from 1 to 10 carbon atoms, and at least one carbon atom of which may be replaced with an entity chosen from oxygen, nitrogen and sulfur atoms and SO₂ groups, and at least one carbon atom of which may be, independently of each other, substituted with at least one halogen atom, with the proviso that T₆ does not comprise any peroxide bonds or diazo, nitro or nitroso radicals;
T₁ or T₅ may also form with T₆ a 5- to 7-membered saturated or unsaturated ring, each ring member being unsubstituted or substituted with one or two radicals R₁₄, which may be identical or different;
two of the adjacent radicals T₁, T₂, T₃, T₄ and T₅ may also form a 5- to 7-membered ring, each ring member being independently represented by a carbon atom which is unsubstituted or substituted with one or two radicals R14, which may be identical or different, a nitrogen atom which is unsubstituted or substituted with a radical R₁₄, an oxygen atom or a sulfur atom;
R₁₀, R₁₁, R₁₂, R₁₃ and R₁₄, which may be identical or different, are chosen from hydrogen atoms, linear and branched, optionally aromatic hydrocarbon-based chains comprising from 1 to 10 carbon atoms, and at least one of the carbon atoms of which may be replaced with an entity chosen from oxygen, nitrogen and sulfur atoms and SO₂ groups, and at least one carbon atom of which may be, independently of each other, substituted with at least one halogen atom, with the proviso that the radical does not comprise any peroxide bonds or diazo, nitro or nitroso radicals;
R₁₀, R₁₁ and R₁₂ may also form, in pairs with the quaternary nitrogen atom to which they are attached, at least one saturated 5- to 7-membered ring, each ring member being independently chosen from carbon atoms that are unsubstituted or substituted with one or two radicals R₁₄, which may be identical or different, nitrogen atoms which are unsubstituted or substituted with a radical R₁₄, oxygen atoms and sulfur atoms;
when n″′ is equal to 0, then the group of formula (VIIIg) may be linked to the compound of formula (VIIIc) and (VIIId) directly via the nitrogen atom of the quaternary ammonium, R₁₃ is then, in this case, a single bond;
V′ is chosen from organic and mineral anions;
Z₂ is chosen from linear and branched C₁-C₁₀ hydrocarbon-based chains, which can form optionally aromatic 5- to 7-membered carbon-based rings, wherein at least one of the carbon atoms may be replaced with at least one entity chosen from oxygen, nitrogen and sulfur atoms and SO₂ groups; with the proviso that the radical Z₂ does not comprise any peroxide bonds, or diazo, nitro or nitroso radicals; and cationic groups Z₃ as defined above, with the proviso that Z₂ is not cationic when R₇, R₉, R_{7′} or R_{9′} is cationic;
A₂ is a carbon-based, pyridyl or pyridazinyl aromatic radical substituted with a 5-membered cationic heteroaromatic radical, optionally substituted with at least one radical R₁₉ of the same definition as R₂, chosen from radicals of formulae (VIIIh) and (VIIIi): 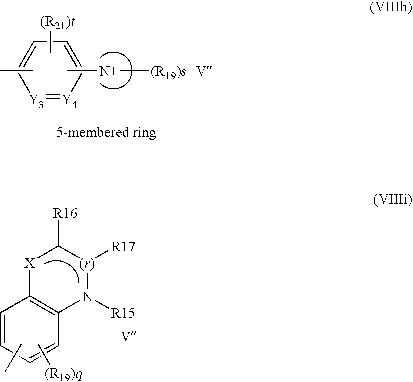
wherein:
r is an integer equal to 0 or 1,
q is an integer equal to 0, 1, 2 or 3,
s is an integer equal to 0, 1, 2, 3, 4 or 5,
t is an integer equal to 0, 1 or 2,
Y₃═Y₄ is chosen from C═C, C═N and N═N,
if r is equal to 0, then X is chosen from O and S atoms, and NR₁₈, and CR₂₀ radicals,
if r is equal to 1, then X is chosen from CR₂₀ radicals.
R₁₅ and R₁₈ have the same definition as R₁ defined above.
R₁₆, R₁₇, R₁₉, R₂₀ and R₂₁, which may be identical or different, are chosen from saturated and unsaturated, linear and branched C₁-C₁₀ hydrocarbon-based chains which can form an optionally aromatic 5- to 7-membered carbon-based ring, wherein at least one of the carbon atoms of the chains may be replaced with an entity chosen from oxygen, nitrogen, halogen and sulfur atoms, and SO₂ groups, with the exception of the carbon linked to the nitrogen atom of the two preceding rings, and with the proviso that the radicals R₁₆, R₁₇, R₁₉, R₂₀ and R₂₁, do not comprise any peroxide bonds, or diazo, nitro or nitroso radicals,
V″ is chosen from organic and mineral anions with the condition that in formula (VIIIa), one of the groups A₁, Z₂ and A₃ is a cationic group;
the compounds of formula (IX)
W₁—N═N—W₂—W₃ (IX)
wherein W₁ is chosen from 5-membered cationic aromatic heterocycle radicals of formula (IXa): 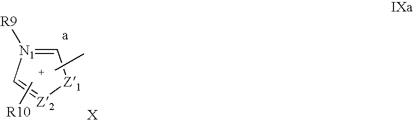
wherein
W₂ is chosen from carbon-based and pyridyl aromatic divalent radicals of formulae (IXb) and (IXc): 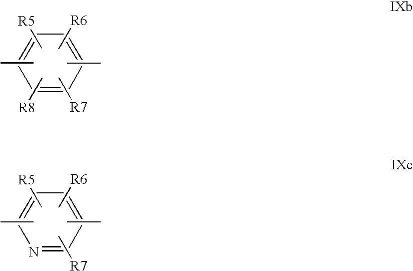
W₃ is chosen from 5- and 6-membered heterocycle radicals of formula (IXd): 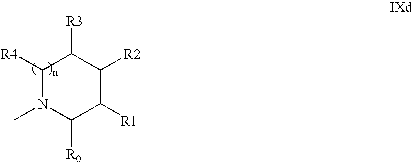
wherein:
Z′₁ is chosen from oxygen and sulfur atoms and NR₁₂ radicals,
Z′₂ is chosen from a nitrogen atom and CR₁₁ radicals,
R₉ and R₁₂, which may be identical or different, are chosen from C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl, sulfonic, and optionally substituted phenyl radicals;
R₁₀ and R₁₁, which may be identical or different, are chosen from hydrogen atoms, and C₁-C₄ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl, sulfonic, optionally substituted phenyl, carboxyl and sulfonylamino radicals,
R₅, R₆, R₇ and R₈, which may be identical or different, are chosen from hydrogen atoms, chlorine atoms, bromine atoms, and linear and branched C₁-C₆ hydrocarbon-based chains, which can form at least one 3- to 6-membered carbon-based ring, and which may be saturated or unsaturated, at least one carbon atom of the carbon-based chain of which may be replaced with an entity chosen from oxygen, nitrogen and sulfur atoms and SO₂ groups, and the carbon atoms of which may be, independently of each other, substituted with at least one halogen atom; with the proviso that R₅, R₆, R₇ and R₈ do not comprise any peroxide bonds or diazo or nitroso radicals;
n is an integer equal to 0 or 1;
R₀, R₁, R₂, R₃ and R₄, which may be identical or different, are chosen from hydrogen atoms, and hydroxyl, amino, acetoxy, and —NR₁₃R₁₄ radicals, R₁₃ and R₁₄, which may be identical or different, are chosen from hydrogen atoms; C₁-C₄ alkyl radicals substituted with at least one entity chosen from halogen atoms, and hydroxyl, C₁-C₂ alkoxy, amino, and C₁-C₂ amino(di)alkyl radicals; sulfonylamino radicals; carboxyl radicals; carboxamido radicals; amido radicals; mono- and dialkylamido radicals; halogen atoms; and C₁-C₆ alkyl radicals substituted with at least one radical chosen from hydroxyl, C₁-C₂ alkoxy, C₂-C₄ (poly)hydroxyalkoxy, amino and C₁-C₂ (di)alkylamino radicals, it being understood that at least one of the groups R₀, R₁, R₂, R₃ and R₄ is other than hydrogen,
X is chosen from organic and mineral anions;
the compounds of formula (X)
W′₁—N═N—W′₂—W′₃ (X)
wherein W′₁ is chosen from 5-membered cationic aromatic heterocycle radicals of formula (Xa): 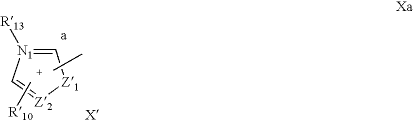
W′₂ is chosen from carbon-based and pyridyl aromatic divalent radicals of formulae (Xb) and (Xc): 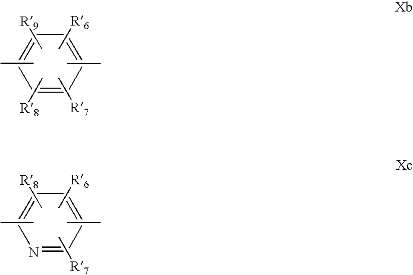
W′₃ is chosen from 7- and 8-membered heterocycle radicals of formula (Xd): 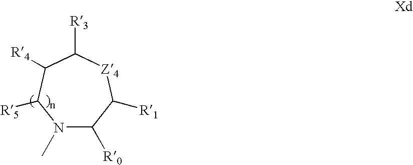
wherein:
Z′₁ is chosen from oxygen and sulfur atoms and NR′₁₂ radicals;
Z′₂ is chosen from a nitrogen atom and CR′₁₁ radicals;
R′₁₂ and R′₁₃, which may be identical or different, are chosen from C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl, and sulfonic radicals; and optionally substituted phenyl radicals;
R′₁₀ and R′₁₁, which may be identical or different, are chosen from hydrogen atoms, C₁-C₄ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; and optionally substituted phenyl radicals; carboxyl radicals; and sulfonylamino radicals;
R′₁₆, R′₁₇, R′₁₈ and R′₉, which may be identical or different, are chosen from hydrogen atoms, chlorine atoms, bromine atoms, linear and branched C₁-C₆ hydrocarbon-based chains, which can form at least one 3- to 6-membered carbon-based ring, and which may be saturated or unsaturated, wherein at least one carbon atom of the carbon-based chain of which may be replaced with an entity chosen from oxygen, nitrogen and sulfur atoms, and SO₂ groups, and the carbon atoms of which may be, independently of each other, substituted with at least one halogen atom; with the proviso that R′₆, R′₇, R′₈ and R′₉ do not comprise any peroxide bonds or diazo or nitroso radicals;
n is an integer equal to 1 or 2;
Z′₄ is chosen from oxygen and sulfur atoms, NR′₂ radicals and CR′₂R″₂ radicals;
R′₀, R′₁, R′₂, R″₂, R′₃, R′₄ and R′₅, which may be identical or different, hydrogen atoms, alkyl radicals, alkoxy radicals, hydroxyl radicals, amino radicals, acetoxy radicals, and —NR₁₄R₁₅ radicals, R₁₄ and R₁₅, which may be identical or different, are chosen from hydrogen atoms, C₁-C₄ alkyl radicals substituted with at least one entity chosen from halogen atoms, and hydroxyl, C₁-C₂ alkoxy, amino and C₁-C₂ amino(di)alkyl radicals; sulfonylamino radicals; carboxyl radicals; carboxamido radicals; amido radicals; mono- and dialkylamido radicals; halogen atoms; and C₁-C₆ alkyl radicals substituted with at least one radical chosen from hydroxyl, C₁-C₂ alkoxy, C₂-C₄ (poly)hydroxyalkoxy, amino, and C₁-C₂ (di)alkylamino radicals,
X′ is chosen from organic and mineral anions;
the compounds of formula (XI) 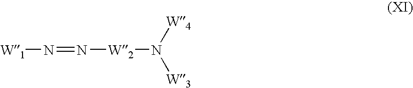
wherein W″₁ is chosen from 5-membered cationic aromatic heterocycle radicals of formula (XIa): 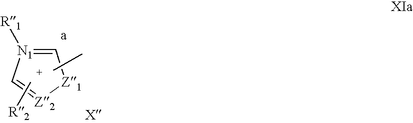
wherein
W″₂ is chosen from carbon-based and pyridyl aromatic divalent radicals of formulae (XIb) and (XIc): 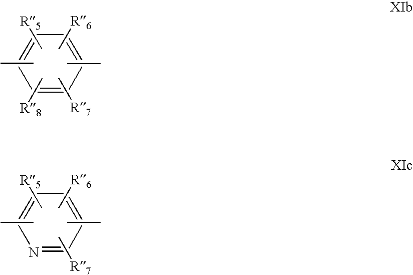
wherein
Z″₁ is chosen from oxygen and sulfur atoms and NR₁₄ radicals,
Z″₂ is chosen from a nitrogen atom CR₁₃ radicals,
R″₁ and R″₄, which may be identical or different, are chosen from C₁-C₈ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; and optionally substituted phenyl radicals;
R″₂ and R″₃, which may be identical or different, are chosen from hydrogen atoms, C₁-C₄ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; optionally substituted phenyl radicals; carboxyl radicals; and sulfonylamino radicals;
R″₅, R″₆, R″₇, R″₈ and W″₄, which may be identical or different, are chosen from hydrogen atoms, chlorine atoms, bromine atoms, and linear and branched C₁-C₆ hydrocarbon-based chains, which can form at least one 3- to 6-membered carbon-based ring, and which may be saturated or unsaturated, wherein at least one carbon atom of the hydrocarbon-based chains may be replaced with an entity chosen from oxygen, nitrogen and sulfur atoms and SO₂ groups, and the carbon atoms of which may be, independently of each other, substituted with at least one halogen atom; with the proviso that R″₅, R″₆, R″₇, R″₈ and W″₄ do not comprise any peroxide bonds, or diazo or nitroso radicals, and W″₄ being a non-aromatic substituent;
W″₃ is chosen from thienyl, pyrazolyl, pyrrolyl, imidazolyl, furyl, triazolyl, thiadiazolyl, isoxazolyl, 5-isothiazolyl, thiazolyl, oxazolyl, pyridyl, pyrimidinyl, triazinyl, pyridazinyl and pyrazinyl radicals, each of these heteroaromatic rings optionally being substituted with at least one C₁-C₄ alkyl radical, optionally substituted with at least one radical chosen from hydroxyl, C₁-C₄ alkoxy, (poly)hydroxyalkoxy, amino, C₁-C₄ (di)alkylamino, C₂-C₄ (poly)hydroxyalkylamino, carboxyl, sulfonyl, alkoxycarbonyl and C₁-C₄ thioether radicals; phenyl radicals optionally substituted with at least one entity chosen from C₁-C₂ alkoxy, amino, C₁-C₂ (di)alkylamino, carboxyl, sulfonyl and C₁-C₄ alkyl radicals, halogen atoms, and C₁-C₂ thioether radicals; halogen atoms; amino radicals; C₁-C₄ alkylamino radicals; C₂-C₄ (poly)hydroxyalkylamino radicals; C₁-C₄ (di)alkylamino radicals; C₁-C₂ alkoxy radicals; carboxyl radicals; and sulfonylamino radicals,
X″ is chosen from organic and mineral anions;
the compounds of formula (XII)
W⁰₁—W⁰₂—N═N—W⁰₃ (XII)
wherien W⁰₁ is chosen from 5-, 6-, 7- and 8-membered heterocycle radicals of formula (XIIa): 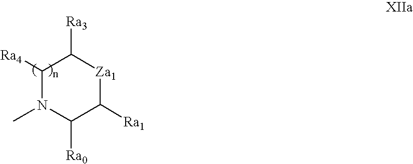
wherein
W⁰₂ is chosen from carbon-based, pyridyl and pyridazinyl aromatic divalent radicals of formula (XIIb): 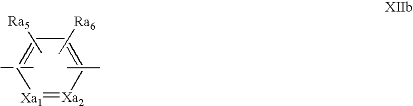
W⁰₃ is chosen from cationic heteroaromatic radicals of formula (XIIc): 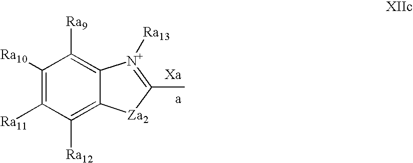
wherein:
n is equal to 0, 1, 2 or 3, it being understood that when n is greater than or equal to 2, then the radicals Ra₄ may be identical or different;
Xa₁ is chosen from a nitrogen atom and CRa₇ radicals;
Xa₂ is chosen from a nitrogen atom and CRa₈ radicals;
Za₁ is chosen from CHRa₂ radicals, oxygen and sulfur atoms, and NRa₁₄ radicals;
Za₂ is chosen from oxygen and sulfur atoms NRa₁₅ radicals;
Ra₀, Ra₁, Ra₂, Ra₃, Ra₄, Ra₅, Ra₆, Ra₇, Ra₈, Ra₉, Ra₁₀, Ra₁₁, and Ra₁₂, which may be identical or different, are chosen from hydrogen atoms, linear and branched C₁-C₁₀ hydrocarbon-based chains, which can form at least one 3- to 6-membered carbon-based ring, and which may be saturated or unsaturated, wherein at least one carbon atom of the carbon-based chains may be replaced with an entity chosen from oxygen, nitrogen and sulfur atoms and SO₂ groups, and the carbon atoms of which may be, independently of each other, substituted with at least one halogen atom, with the proviso that Ra₀, Ra₁, Ra₂, Ra₃, Ra₄, Ra₅, Ra₆, Ra₇, Ra₈, Ra₉, Ra₁₀, Ra₁₁, and Ra₁₂ do not comprise any peroxide bonds, or diazo or nitroso radicals;
Ra₁₄ is chosen from a hydrogen atom, and linear and branched C₁-C₁₀ hydrocarbon-based chains, which can form at least one 3- to 6-membered carbon-based ring, and which may be saturated or unsaturated, wherein at least one carbon atom of the carbon-based chains may be replaced with an entity chosen from oxygen, nitrogen and sulfur atoms and SO₂ groups, and the carbon atoms of which may be, independently of each other, substituted with at least one halogen atom, with the proviso that Ra₁₄ does not comprise any peroxide bonds, or diazo or nitroso radicals, it being understood that the said oxygen, nitrogen and sulfur atoms are not directly linked to the nitrogen atom bearing the radical Ra₁₄;
Ra₅ and Ra₆ may form a carbon-based aromatic ring, such as a phenyl;
Ra13 and Ra15, which may be identical or different, are chosen from C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl, sulfonic and optionally substituted phenyl radicals;
the bond a of the cationic ring of formula (XIIc) is linked to the azo group of formula (XIIa),
Xa is chosen from organic and mineral anions;
the compounds of formula (XIII)
A-N═N—B (XIII)
wherein A is chosen from the radicals A₁ to A₃: 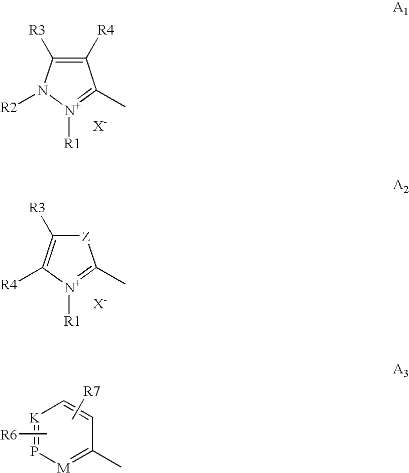
wherein, R₁ is chosen from C₁-C₄ alkyl radicals, phenyl radicals that may be substituted with a C₁-C₄ alkyl radical or a halogen atom chosen from chlorine, bromine, iodine and fluorine;
R₂ is chosen from C₁-C₄ alkyl radicals and phenyl radicals;
R₃ and R₄, which may be identical or different, are chosen from C₁-C₄ alkyl radicals, and phenyl radicals, or alternatively, in the case of the structure A₁, may together form a substituted benzene ring, and in the case of the structure A₂, may together form a benzene ring optionally substituted with at least one radical chosen from C₁-C₄ alkyl, C₁-C₄ alkoxy and NO₂ radicals;
R₃ may also be a hydrogen atom;
Z is chosen from oxygen and sulfur atoms and —NR₂ radicals;
M is chosen from —CH, —CR, wherein R is a C₁-C₄ alkyl, and —NR₅ (X⁻)ᵣ radicals;
K is chosen from —CH, —CR, wherein R is a C₁-C₄ alkyl, and —NR₅ (X⁻)ᵣ radicals;
P is chosen from —CH, —CR, wherein R is a C₁-C₄ alkyl, and —NR₅ (X⁻)ᵣ radicals r is equal to 0 or 1;
R₅ is chosen from O⁻ atoms, C₁-C₄ alkoxy radicals, and C₁-C₄ alkyl radicals;
R₆ and R₇, which may be identical or different, are chosen from hydrogen atoms, halogen atoms chosen from chlorine, bromine, iodine and fluorine, and C₁-C₄ alkyl, C₁-C₄ alkoxy, and NO₂ radicals;
X⁻ is an anion, with the proviso that,
if R₄ is a C₁-C₄ alkyl radical and Z is a sulfur atom, then R₃ is not a hydrogen atom
if R₅ is an O⁻ atom, then r is equal to zero.
if K or P or M is the radical —N-alkyl C₁-C₄ X⁻, then R₆ or R₇ is not a hydrogen atom.
if K is the radical —NR₅(X⁻)ᵣ, then M is the same as P, and are chosen from —CH and —CR radicals,
if M is the radical —NR₅(X⁻)ᵣ, then K is the same as P, and are chosen from —CH and —CR radicals,
if P is the radical —NR₅(X⁻)ᵣ, then K is the same as M, and are chosen from —CH and —CR radicals,
if Z is the radical —NR₂, and R₂ is a C₁-C₄ alkyl radical, then at least one of the radicals R₁, R₃ or R₄ of A₂ is not a C₁-C₄ alkyl radical,
B is chosen from either:
a group of formula B₁: 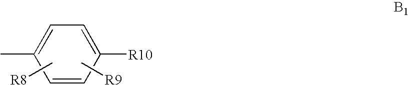
wherein, R₈ is chosen from a hydrogen atom, halogen atoms chosen from chlorine, bromine, iodine and fluorine, C₁-C₄ alkyl radicals, C₁-C₄ alkoxy radicals, —OH radicals, —NO₂ radicals, —NHR₁₁ radicals, —NR₁₂R₁₃ radicals and —NHCOC₁-C₄ alkyl radicals, or forms with R₉ a 5- or 6-membered ring, optionally comprising at least one hetero atom chosen from nitrogen, oxygen and sulfur;
R₉ is chosen from a hydrogen atom, halogen atoms chosen from chlorine, bromine, iodine and fluorine, C₁-C₄ alkyl and C₁-C₄ alkoxy radicals, or forms with R₁₀ or R₁₁ a 5- or 6-membered ring optionally comprising at least one hetero atom chosen from nitrogen, oxygen and sulfur;
R₁₀ is chosen from a hydrogen atom, —OH radicals, —NHR₁₁ radicals, and —NR₁₂R₁₃. radicals;
R₁₁ is chosen from a hydrogen atom, C₁-C₄ alkyl radicals, C₁-C₄ monohydroxyalkyl radicals, C₂-C₄ polyhydroxyalkyl radicals, and phenyl radicals,
R₁₂ and R₁₃, which may be identical or different, are chosen from C₁-C₄ alkyl radicals, C₁-C₄ monohydroxyalkyl radicals, and C₂-C₄ polyhydroxyalkyl radicals; or
5- and 6-membered nitrogenous heterocyclic groups which may comprise other hetero atoms and/or carbonyl groups and which may be substituted with at least one radical chosen from alkyl C₁-C₄, amino and phenyl radicals;
the compounds of formula (XIV):
W₁—N═N—W₂—W₃ (XIV)
wherein
W₁ is chosen from 5-membered cationic aromatic heterocycle radicals of formula (II): 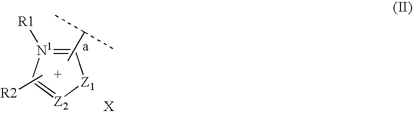
W₂ is chosen from carbon-based and pyridyl aromatic divalent groups of formulae (IV) and (V): 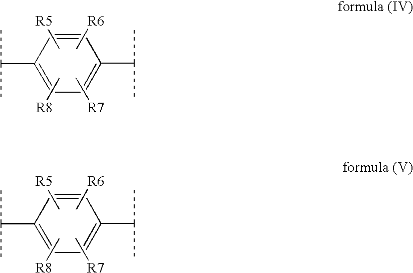
W₃ is chosen from 5- and 6-membered nitrogenous heteroaromatic radicals linked to W₂ via the nitrogen atom of the ring of the heteroaromatic radical, the heteroaromatic radical being chosen from pyrazolyl, pyrrolyl, imidazolyl, triazolyl, thiadiazolyl, pyridazinyl and pyrazinyl radicals, each of these heteroaromatic radicals being optionally substituted with at least one entity chosen from hydrogen atoms; C₁-C₆ alkyl radicals, optionally substituted with at least one radical chosen from hydroxyl, C₁-C₄ alkoxy, (poly)hydroxyalkoxy, amino, C₁-C₄ (di)alkylamino, C₂-C₄ (poly)hydroxyalkylamino, carboxyl, sulfonyl, alkoxycarbonyl and C₁-C₄ thioether radicals; phenyl radicals optionally substituted with at least one entity chosen from halogen atoms, and C₁-C₂ alkoxy, amino, C₁-C₂ (di)alkylamino, carboxyl, sulfonyl, C₁-C₄ alkyl, and C₁-C₂ thioether radicals,
Z₁ is chosen from oxygen and sulfur atoms and NR₄ radicals,
Z₂ is chosen from a nitrogen atom and CR3 radicals,
R1 and R4, which may be identical or different, are chosen from C₁-C₈ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl, and sulfonic radicals; and optionally substituted phenyl radicals,
R2 and R3, which may be identical or different, are chosen from hydrogen atoms; C₁-C₆ alkyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl, and sulfonic radicals; optionally substituted phenyl radicals; carboxyl radicals; and sulfonylamino radicals;
R5, R6, R7 and R8, which may be identical or different, are chosen from hydrogen atoms; chlorine atoms; bromine atoms; and linear and branched C₁-C₆ hydrocarbon-based chains, which can form at least one 3- to 6-membered carbon-based ring, and which may be saturated or unsaturated, wherein at least one carbon atom of the carbon-based chains may be replaced with an entity chosen from oxygen, nitrogen and sulfur atoms and SO₂ groups, and the carbon atoms of which may be, independently of each other, substituted with at least one halogen atom; with the proviso that R5, R6, R7 and R8 do not comprising any peroxide bonds, or diazo or nitroso radicals,
X is chosen from organic and mineral anions;
the compounds of formula (XV):
W₁—N═N—W₂—(W₃)ₙ—W₄—N═N—W₅ (XV)
wherein
n is equal to 0 or 1,
W1 and W5, which may be identical or different, are chosen from heteroaromatic radicals of formulae (II) and (III): 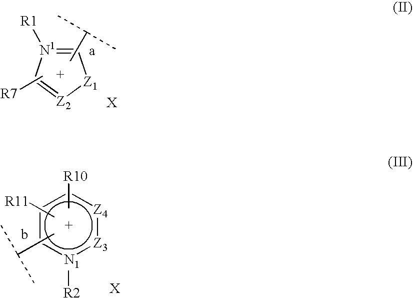
W3 is chosen from an oxygen atom, NR14 radicals, —NR15—W6-NR16— radicals, —NR16—W6-O— radicals, —O—W6-O— radicals, W6 radicals, and carbonyl radicals,
W2 and W4, which can be identical or different, are chosen from carbon-based, pyridyl and pyridazinyl aromatic radicals of formula (IV) 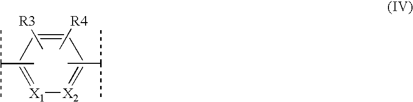
W6 is chosen from 5- and 6-membered aromatic and heteroaromatic groups of formula (V): 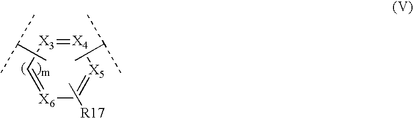
wherein
m is equal to 0 or 1,
X1 is chosen from a nitrogen atom and CR5 radicals,
X2 is chosen from a nitrogen atom and CR6 radicals,
X3 is chosen from nitrogen and carbon atoms and CR18 radicals,
X4 is chosen from nitrogen and carbon atoms and CR19 radicals,
X5 is chosen from nitrogen and carbon atoms and CR20 radicals,
X6 is chosen from nitrogen and carbon atoms and CR21 radicals,
Z1 is chosen from oxygen and sulfur atoms NR8 radicals,
Z2 is chosen from a nitrogen atom and CR9 radicals,
Z3 is chosen from a nitrogen atom and CR12 radicals,
Z4 is chosen from a nitrogen atom and CR13 radicals,
the bond a of the 5-membered cationic ring of formula (II) is linked to the azo group of formula (XV),
the bond b of the 6-membered cationic ring of formula (III) is linked to the azo group of formula (XV),
it being understood that when X3, X4, X5, and X6 represent a carbon atom, then they are linked to W2 or W4,
it being understood that formula (V) does not comprise more than three nitrogen atoms,
it being understood that when formula (V) comprises three nitrogen atoms, they are not contiguous,
R1, R2 and R8, which may be identical or different, are chosen from linear and branched C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; phenyl radicals optionally substituted with at least one radicals chosen from hydroxyl, C₁-C₂ alkoxy, C₂-C₄ (poly)hydroxyalkoxy, amino, C₁-C₂ (di)alkylamino, carboxyl and sulfonic radicals; and halogen atoms;
R3, R4, R5, R6, R7, R9, R10, R11, R12, R13, R14, R15, R16, R17, R18, R19, R20 and R21, which may be identical or different, are chosen from hydrogen atoms, linear and branched C₁-C₁₆ hydrocarbon-based chains, which may be saturated or unsaturated, wherein at least one carbon atom of the carbon-based chains of which may be replaced with an entity chosen from oxygen, nitrogen and sulfur atoms and SO₂ groups, and the carbon atoms of which may be, independently of each other, substituted with one or more halogen atoms; with the proviso that R3, R4, R5, R6, R7, R9, R10, R11, R12, R13, R14, R15, R16, R17, R18, R19, R20 and R21 do not comprise any peroxide bonds, or diazo or nitroso radicals,
R7 with R9, R10 with R11 and R12 with R13 may also form a carbon-based aromatic ring,
X is chosen from organic and mineral anions;
the compounds of formula (XVI):
[W₁—N═N—W₂—W₃—(W₄—W₅)ₚ]₂-L (XVI)
wherein
p is equal to 0 or 1.
W1 is chosen from heteroaromatic radicals of formulae (II) and (III): 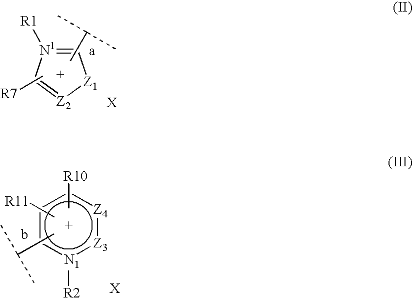
W2 is chosen from carbon-based, pyridyl and pyridazinyl aromatic groups of formula (IV): 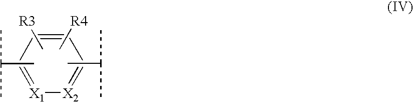
W3 and W5, which may be identical or different, are chosen from —NR14— radicals, oxygen atoms, and nitrogen atoms,
W4 is chosen from linear and branched C₁-C₁₆ hydrocarbon-based chains, which can form at least one 3- to 6-membered carbon-based ring, and which may be saturated or unsaturated, wherein at least one carbon atom of the carbon-based chains of which may be replaced with an entity chosen from oxygen, nitrogen and sulfur atoms and SO₂ groups, and the carbon atoms of which may be, independently of each other, substituted with at least one halogen atom, with the proviso that W4 does not comprise any peroxide bonds, or diazo or nitroso radicals; piperazine rings; and radicals of formula (V): 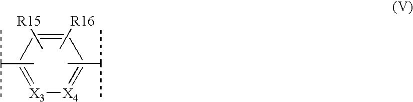
wherein:
X1 is chosen from a nitrogen atom and CR5 radicals
X2 is chosen from a nitrogen atom and CR6 radicals
X3 is chosen from a nitrogen atom and CR17 radicals
X4 is chosen from a nitrogen atom and CR18 radicals
Z1 is chosen from oxygen and sulfur atoms and NR8 radicals,
Z2 is chosen from a nitrogen atom and CR9 radicals,
it being understood that (Z1, Z2) is other than (NR8, CR9),
Z3 is chosen from a nitrogen atom and CR12 radicals,
Z4 is chosen from a nitrogen atom and CR13 radicals,
the bond a of the 5-membered cationic ring of formula (II) is linked to the azo group of formula (XVI),
the bond b of the 6-membered cationic ring of formula (III) is linked to the azo group of formula (XVI),
when p is equal to 0, then L is chosen from linear and branched C₁-C₁₆ hydrocarbon-based chains, which can form at least one 3- to 6-membered carbon-based ring, at least one carbon atom of the carbon-based chains of which may be replaced with an entity chosen from oxygen, nitrogen and sulfur atom and SO₂ groups, and the carbon atoms of which may be, independently of each other, substituted with at least one halogen atoms; with the proviso that L does not comprise any peroxide bonds, or diazo or nitroso radicals,
when p is equal to 1, then L is chosen from linear and branched C₁-C₁₆ hydrocarbon-based chains, which can form at least one 3- to 6-membered carbon-based ring, and which may be saturated or unsaturated, wherein at least one carbon atom of the carbon-based chains of which may be replaced with an entity chosen from oxygen, nitrogen and sulfur atoms and SO₂ groups, with the proviso that L does not comprise any peroxide bonds, or diazo or nitroso radicals,
R3, R4, R5, R6, R7, R9, R10, R11, R12, R13, R14, R15, R16, R17 and R18, which may be identical or different, are chosen from hydrogen atoms, linear and branched C₁-C₁₆ hydrocarbon-based chains, which can form at least one 3- to 6-membered carbon-based ring, and which may be saturated or unsaturated, wherein at least one carbon atom of the carbon-based chains may be replaced with an entity chosen from oxygen, nitrogen and sulfur atoms and SO₂ groups, and the carbon atoms of which may be, independently of each other, substituted with at least one halogen atom; with the proviso that R3, R4, R5, R6, R7, R9, R10, R11, R12, R13, R14, R15, R16, R17 and R18 do not comprise any peroxide bonds, or diazo or nitroso radicals,
R3 with R4, R7 with R9, R10 with R11, R12 with R13 and R15 with R16 may also form a carbon-based aromatic ring,
R1, R2, and R8 are chosen from linear and branched C₁-C₈ alkyl radicals, optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals; and phenyl radicals optionally substituted with at least one radical chosen from hydroxyl, C₁-C₂ alkoxy, C₂-C₄ (poly)hydroxyalkoxy, amino, C₁-C₂ (di)alkylamino, carboxyl and sulfonic radicals,
X is chosen from organic and mineral anions;
the compounds of formula (XVII):
W₁—N═N—W₂—NW₃—W₄—W₅ (XVII)
wherein
W1 is chosen from 5- and 6-membered cationic aromatic heterocycle radicals of formulae (II) and (III): 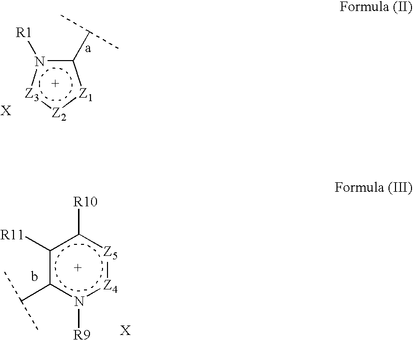
wherein:
Z1 is chosen from oxygen and sulfur atoms, NR2 radicals and CR3 radicals,
Z2 is chosen from a nitrogen atom and CR4 radicals,
Z3 is chosen from NR12 and CR13 radicals,
Z4 is chosen from a nitrogen atom and CR14 radicals,
Z5 is chosen from nitrogen atom and CR15 radicals,
the bond a links the 5-membered cationic ring of formula (II) to the azo functional group of formula (XVII).
the bond b of the 6-membered cationic ring of formula (III) is linked to the azo functional group of formula (XVII).
X is chosen from organic and mineral anions;
W2 and W4, which may be identical or different, are chosen from carbon-based and pyridyl aromatic divalent radicals of formulae (IV) and (V): 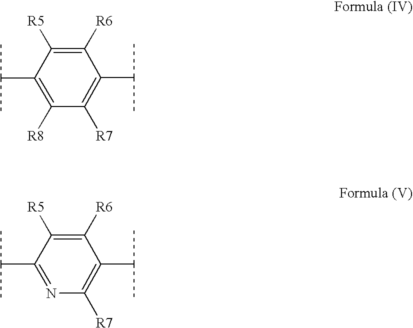
W3 is chosen from a hydrogen atom and C₁-C₆ alkyl radicals optionally substituted with at least one radical chosen from hydroxyl radicals, alkoxy radicals, amino radicals, monoalkylamino radicals, and dialkylamino radicals,
W5 is chosen from 5-membered nitrogenous heteroaromatic radicals linked to W4 via the nitrogen atom of the ring of the heteroaromatic radical, this heteroaromatic radical being chosen from pyrazolyl, pyrrolyl, imidazolyl, triazolyl and thiadiazolyl radicals, each of these heteroaromatic radicals possibly being substituted with at least one entity chosen from hydrogen, chlorine and fluorine atoms; C₁-C₆ alkyl radicals optionally substituted with at least one radical chosen from hydroxyl, C₁-C₄ alkoxy, (poly)hydroxyalkoxy, amino, C₁-C₄ (di)alkylamino, C₂-C₄ (poly)hydroxyalkylamino, carboxyl, sulfonyl, alkoxycarbonyl and C₁-C₄ thioether radicals; and phenyl radicals optionally substituted with at least one radical chosen from hydroxyl, C₁-C₂ alkoxy, amino, C₁-C₂ (di)alkylamino, carboxyl, sulfonyl, C₁-C₄ alkyl, halogen and C₁-C₂ thioether radicals,
R1, R2, R9 and R12, which may be identical or different, are chosen from optionally substituted phenyl radicals and C₁-C₈ alkyl radicals optionally substituted with at least one radical chosen from hydroxyl, C₁-C₂ alkoxy, C₂-C₄ (poly)hydroxyalkoxy, amino and C₁-C₂ (di)alkylamino radicals,
R5, R6, R7 and R8, which may be identical or different, are chosen from hydrogen atoms; chlorine atoms; bromine atoms; and linear and branched C₁-C₈ hydrocarbon-based chains, which can form at least one 3- to 6-membered carbon-based ring, and which may be saturated or unsaturated, wherein at least one carbon atom of the carbon-based chains may be replaced with an entity chosen from oxygen, nitrogen and sulfur atoms and SO₂ groups, and the carbon atoms of which may be, independently of each other, substituted with at least one halogen atoms; with the proviso that R5, R6, R7 and R8 do not comprise any peroxide bonds, or diazo or nitroso radicals,
R3, R4, R10, R11, R13, R14 and R15, which may be identical or different, are chosen from hydrogen atoms, and linear and branched C₁-C₁₆ hydrocarbon-based chains, which can form at least one 3- to 6-membered carbon-based ring, and which may be saturated or unsaturated, wherein at least one carbon atom of the carbon-based chains of which may be replaced with an entity chosen from oxygen, nitrogen and sulfur atoms and SO₂ groups, and the carbon atoms of which may be, independently of each other, substituted with at least one halogen atom; with the proviso that R3, R4, R10, R11, R13, R14 and R15 do not comprise any peroxide bonds, or diazo or nitroso radicals,
R4 with R13 and R14 with R15 may also form a carbon-based aromatic ring.

3. The composition according to claim 2, wherein the azo, methine and azomethine cationic direct dyes are chosen from the compounds of formulae I, II, III and III′.

4. The composition according to claim 1, wherein the at least one cationic direct dye is present in an amount ranging from 0.0005% to 15% by weight, relative to the total weight of the composition.

5. The composition according to claim 1, wherein the at least one alcohol oxidase enzyme is chosen from those of category EC 1.1.3.

6. The composition according to claim 5, wherein the at least one alcohol oxidase enzyme is chosen from primary alcohol oxidases (EC 1.1.3.13), secondary alcohol oxidases (EC 1.1.3.18), long hydrocarbon chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7).

7. The composition according to claim 6, wherein the at least one alcohol oxidase enzyme is derived from a species chosen from: Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia sp. (pastoris, methanolica, angusta) and Candida sp. (boidinii, albicans, tropicalis), Pinus strobus, Gastropode mollusc, and Manduca sexta.

8. The composition according to claim 7, wherein the at least one alcohol oxidase enzyme is Pichia pastoris.

9. The composition according to claim 1, wherein the at least one alcohol oxidase enzyme is present in an amount ranging from 0.05% to 20% by weight, relative to the total weight of the composition.

10. The composition according to claim 1, wherein the at least one alcohol oxidase enzyme is present in an amount ranging from 10³ U to 10⁵ U, per 100 g of the dye composition.

11. The composition according to claim 1, wherein the at least one substrate for the enzyme is an alcohol chosen from branched and unbranched, saturated and unsaturated, substituted and unsubstituted primary and secondary alcohols, long hydrocarbon chain alcohols and aromatic alcohols.

12. The composition according to claim 11, wherein the at least one substrate for the enzyme is present in an amount ranging from 0.01% to 60% by weight, relative to the total weight of the composition.

13. The composition according to claim 1, wherein the at least one oxidation dye precursor is a standard oxidation base chosen from para-phenylenediamines, bis(phenyl)alkylenediamines, para-aminophenols, ortho-aminophenols, heterocyclic bases, and the addition salts thereof.

14. The composition according to claim 13, wherein the at least one oxidation base is present in an amount ranging from 0.0001% to 20% by weight, relative to the total weight of the composition.

15. The composition according to claim 1, wherein the at least one oxidation dye precursor is a standard oxidation coupler chosen from meta-phenylenediamines, meta-aminophenols, meta-diphenols, naphthalene-based couplers, heterocyclic couplers, and the addition salts thereof.

16. The composition according to claim 15, wherein the at least one coupler is present in an amount ranging from 0.0001% to 20% by weight, relative to the total weight of the composition.

17. The composition according to claim 1, further comprising at least one direct dye, other than azo, methine or azomethine cationic direct dyes.

18. A process for dyeing keratin fibers, comprising
applying to the fibers at least one dye composition comprising, in a medium that is suitable for dyeing, at least one oxidation dye precursor; at least one alcohol oxidase enzyme; at least one substrate bearing an alcohol functional group for the enzyme, and at least one cationic direct dye chosen from azo, methine and azomethine cationic direct dyes, wherein the at least one substrate is optionally totally or partially substituted with the oxidation dye precursor if the at least one precursor bears at least one functional group chosen from aliphatic and aromatic alcohol functional groups, and
leaving the composition on the fibers for a time that is sufficient to develop a desired coloration.

19. The process according to claim 18, wherein the dye composition is a ready-to-use composition, the process further comprising storing the at least one dye composition in anaerobic form, free of oxygen gas.

20. The process according to claim 18, further comprising
storing separately at least one composition (A) comprising, in a medium that is suitable for dyeing keratin fibers, at least one oxidation dye precursor, and at least one composition (B) comprising, in a medium that is suitable for dyeing keratin fibers, at least one alcohol oxidase enzyme, wherein the composition (A) and/or the composition (B) comprise at least one substrate for the enzyme and the composition (A) and/or the composition (B) comprise at least one cationic dye chosen from azo, methine and azomethine cationic direct dyes, and
mixing together the compositions (A) and (B) at the time of use before applying this mixture to the keratin fibers.

21. The process according to claim 18, further comprising
separately storing at least one composition (A) comprising, in a medium that is suitable for dyeing keratin fibers, at least one oxidation dye precursor, at least one substrate for the alcohol oxidase enzyme and at least one cationic direct dye chosen from azo, methine and azomethine cationic direct dyes at least one composition (B) comprising, in a medium that is suitable for dyeing keratin fibers, at least one alcohol oxidase enzyme, and
mixing together the compositions (A) and (B) at the time of use, before applying this mixture to the keratin fibers.

22. A multi-compartment dyeing kit, comprising at least one first compartment comprising the composition (A) comprising, in a medium that is suitable for dyeing keratin fibers, at least one oxidation dye precursor, and at least one second compartment comprising the composition (B) comprising, in a medium that is suitable for dyeing keratin fibers, at least one alcohol oxidase enzyme,
wherein the composition (A) and/or the composition (B) comprise at least one substrate for the enzyme and the composition (A) and/or the composition (B) comprise at least one cationic dye chosen from azo, methine and azomethine cationic direct dyes.
